(19) United States
(12) Patent Application Publication
(10) Pub. No.: US 2024/0182602 A1
(43) Pub. Date: Jun. 6, 2024

(54) INHIBITED STARCHES, METHODS FOR MAKING AND USING THEM, AND EMULSIONS AND FOAMS INCLUDING THEM

(71) Applicant: TATE & LYLE SOLUTIONS USA LLC, Hoffman Estates, IL (US)

(72) Inventors: Weichang Liu, Hoffman Estates, IL (US); Judith K. Whaley, Hoffman Estates, IL (US); Ehsan Jenab, Hoffman Estates, IL (US)

(21) Appl. No.: 18/553,332

(22) PCT Filed: Mar. 29, 2022
(86) PCT No.: PCT/US2022/022342

§ 371 Date: Sep. 29, 2023

Related U.S. Application Data
(60) Provisional application No. 63/168,865, filed on Mar. 31, 2021.

(51) Int. Cl. C08B 30/00; A61K 8/04; A61K 8/06; A61K 8/73; A61K 47/36; A61Q 19/00; C08L 3/02

(52) U.S. Cl. CPC C08B 30/00; A61K 8/046; A61K 8/06; A61K 8/732; A61K 47/36; A61Q 19/00; C08L 3/02; A61K 2800/10; A61K 2800/654

(57) ABSTRACT

The present disclosure provides inhibited starches, methods of making inhibited starches, and emulsions and foams including them, and food and beverage products including them.

### CROSS-REFERENCE TO RELATED APPLICATIONS

This application claims the benefit of priority of U.S. Provisional Patent Application No. 63/168,865, filed Mar. 31, 2021, which is hereby incorporated by reference herein in its entirety.

### BACKGROUND OF THE DISCLOSURE

### Field of the Disclosure

The present disclosure relates generally to inhibited starch products. More particularly, the present disclosure relates to inhibited starches useful in stabilizing emulsions and foams.

### Technical Background

Emulsions are mixtures of two or more immiscible phases in which one is dispersed into the other in the form of small droplets. One common form of an emulsion is oil droplets dispersed in a continuous aqueous phase. Foams can be thought of as replacing the dispersed droplets of an emulsion with bubbles of a gas, e.g., air, but the principle is the same—in each, droplets or bubbles need to be stabilized to prevent them from re-coalescing.

Synthetic surfactants, adsorbed to the interface of the two phases, typically have been used to increase the stability of emulsions and foams by, e.g., decreasing the interfacial tension, among other physical principles. Proteins have also been used as emulsifiers in food emulsions, as well as a small number of polysaccharides, like gum Arabic, modified celluloses and some starches. However, starch is usually gelatinized and/or dissolved in food systems, and so such compositions typically support emulsions and foams only poorly or modestly.

Emulsions stabilized by small solid particles are known as Pickering emulsions. The particles (e.g., colloidal silica, titanium oxide or clays, latex, fat crystals, aggregated proteins, cocoa powder and others) are also able to adsorb onto the interface between the two phases to stabilize the emulsion and/or foam. Properties such as hydrophobicity, shape, and size of the particle can influence the stability of the emulsion, but Pickering emulsions can display extreme long-term stability.

There is a need for highly stable emulsions and foams in the food and personal care industries that derive from natural and sustainable products yet offer the stability of Pickering particulate emulsifiers.

### SUMMARY OF THE DISCLOSURE

One aspect of the disclosure is an inhibited starch product having a protein content in the range of 0.2-8 wt % on a dry starch basis.

Another aspect of the disclosure is an inhibited starch product having a protein content in the range of 0.2-8 wt % on a dry starch basis and a median primary particle size in the range of 0.2-5 microns.

Another aspect of the disclosure is an inhibited starch product having a protein content in the range of 0.2-8 wt % on a dry starch basis and a sedimentation volume in the range of 5-50 mL/g.

Another aspect of the disclosure is an inhibited starch product having a protein content in the range of 0.2-8 wt % on a dry starch basis, a median primary particle size in the range of 0.2-5 microns, and a sedimentation volume in the range of 5-50 mL/g.

Another aspect of the disclosure is an emulsion comprising an emulsified phase that is a hydrophobic phase emulsified within a hydrophilic phase, stabilized by a gelatinized inhibited starch product as described herein.

Another aspect of the disclosure is an emulsion comprising an emulsified phase that is a hydrophobic phase emulsified within a hydrophilic phase, stabilized by a gelatinized inhibited starch, wherein the gelatinized inhibited starch has a median primary particle size in the range of 0.2-5 microns and a protein content in the range of 0.2-8 wt. %.

Another aspect of the disclosure is a foam comprising gas bubbles stabilized within a liquid phase, stabilized by a gelatinized inhibited starch product as described herein.

Another aspect of the disclosure is a foam comprising gas bubbles stabilized within a liquid phase, stabilized by a gelatinized inhibited starch, wherein the gelatinized inhibited starch has a median primary particle size in the range of 0.2-5 microns and a protein content in the range of 0.2-8 wt.

Another aspect of the disclosure is a food or beverage product comprising an emulsion or foam according to other aspects of the disclosure.

Another aspect of the disclosure is a method for making an emulsion or a product including the emulsion, the method comprising mixing under shear a hydrophobic phase, a hydrophilic phase and an inhibited starch product according to another aspect of the disclosure.

Another aspect of the disclosure is a method for making an emulsion or a product including the emulsion, the method comprising mixing under shear a hydrophobic phase, a hydrophilic phase and an unfragmented inhibited starch having a sedimentation volume in the range of 5-50 mL/g and a protein content in the range of 0.2-8 wt. % under conditions sufficient to fragment the starch to a median primary particle size in the range of 0.2-5 microns and to form the emulsion (e.g., under shear).

Another aspect of the disclosure is a method for making a foam or a product including the foam, the method including mixing the liquid phase and an inhibited starch as described herein under conditions sufficient to form the foam (e.g., under shear and/or with addition of gas).

Another aspect of the disclosure is a method for making a foam or a product including the foam, the method comprising mixing the liquid and an unfragmented inhibited starch having a sedimentation volume in the range of 5-50 mL/g and a protein content in the range of 0.2-8 wt. % under conditions sufficient to fragment the starch to a median primary particle size in the range of 0.2-5 microns and to form the foam (e.g., under shear and/or with addition of gas).

Other aspects of the disclosure will be evident for the detailed description provided herein.

Additional aspects of the disclosure will be evident from the disclosure herein.

### BRIEF DESCRIPTION OF THE DRAWINGS

FIGS. 1A to 1D present scanning electron micrograph (SEM) images of amaranth starch samples. FIG. 1A and FIG. 1B show scans of S/L-5, while FIG. 1C and FIG. 1D show scans of S/L-1.

FIG. 2 presents a graph showing RVA plot of native (S/L-1) and inhibited (S/L-2, S/L-3) amaranth starches.

FIGS. 3A-3B are pictures of creamed emulsions after one week. (A): from left to right, 1% uncooked inhibited starch (S/L-2), 1% cooked inhibited starch (S/L-2), 2% uncooked inhibited starch (S/L-2), 2% cooked inhibited starch (S/L-2), 4% uncooked inhibited starch (S/L-2); and (b): from left to right, 1% uncooked native starch (S/L-1), 2% uncooked native starch (S/L-1), 4% uncooked native starch (S/L-1).

FIGS. 4A-4D are a series of micrographs of emulsion droplets stabilized by uncooked native amaranth starch at 2% starch content.

FIGS. 5A-5D are a series of micrographs of emulsion droplets stabilized by uncooked native amaranth starch at 4% starch content.

FIG. 6 is a photograph of creamed emulsions after 1.5 hours of rest time.

FIGS. 7A-7D are another series of micrographs of emulsion droplets stabilized by cooked inhibited amaranth starch at 1% starch content.

FIGS. 8A-8D are a series of micrographs of emulsion droplets stabilized by cooked inhibited amaranth starch at 2% starch content.

FIGS. 9A-9D are a series of micrographs of emulsion droplets stabilized by cooked inhibited amaranth starch at 4% starch content.

FIG. 10 is a graph of the emulsion index of emulsions made from native and inhibited emulsions.

FIG. 11 is a graph of the stability of foam stabilized by cooked inhibited amaranth starch.

FIG. 12 presents photographs of a foam stabilized by cooked inhibited amaranth starch at 5 minutes (L) and 4 hours (R) after foam preparation.

FIGS. 13A-13B are scanning electron microscope images of quinoa starch at 500× magnification (A) and 1,500× magnification (B).

FIG. 14 is a diagram of the RVA curve of heat treated quinoa starch.

FIG. 15 is a diagram of the emulsion index of heat-treated quinoa starch at different starch concentrations.

FIGS. 16A-16C are a series of micrographs of emulsion droplets of heat-treated quinoa starch (S/L-5) after two days at 1%, 2% and 4% concentration, respectively.

FIG. 17 is a graph illustrating foam stability of heat-treated quinoa starch (S/L-5) at 2% concentration.

FIG. 18 is a photograph of foams stabilized by 2% quinoa starch after 24 hours storage.

FIGS. 19A, 19B and 19C are micrographs of inhibited corn starches under varying degrees of shear.

FIGS. 20A and 20B are graphs of, respectively, mean and mode droplet sizes of emulsions made with fragmented inhibited corn starches.

FIGS. 21A and 21B are micrographs of emulsions made with fragmented inhibited corn starches.

FIG. 22 is a set of graphs of emulsion index data for emulsions made with fragmented inhibited corn starches.

FIGS. 23A, 23B, 23C and 23D are graphs of, respectively, mean, mode d50 and d90 droplet sizes of emulsions made with fragmented inhibited corn starches.

### DETAILED DESCRIPTION

The present inventors have unexpectedly determined that an inhibited starch having a small particle size and a relatively high protein content in the range of 0.2-8 wt % can stabilize emulsions and foams and exhibit high emulsion and/or foam stability, even in cases of high or low pH, high ionic strength, high temperature and shear abuse during shipping and storage. Notably, such starch products can stabilize emulsions even after being cooked, e.g., in the processing of a food or beverage product. This is in contrast to conventional native starches used in Pickering emulsions.

The present inventors have noted that such starch-stabilized emulsions and foams can be provided in a number of fashions. For example, an inhibited starch having a relatively high protein content can itself have a small particle size, e.g., by being provided from small granule-size starches, or by being fragmented during production. Such a starch product can be used to make an emulsion or foam. The inventors also contemplate that the starch product used as an ingredient is not itself of a small particle size, but rather is fragmented to small particle size during the emulsification or foaming process. Accordingly, a variety of different starch products are suitable for use as ingredients in making stabilized emulsions and foams according to the present disclosure.

One aspect of the disclosure is an inhibited starch having a protein content in the range of 0.2-8 wt % on a dry starch basis. As described below, in many embodiments, such a starch product itself has a small particle size suitable for stabilization of emulsions and foams. For example, in various embodiments as otherwise described herein, the inhibited starch has a median primary particle size in the range of 0.2-5 microns.

There are a variety of starch sources that may be utilized in the methods and the products of the present disclosure. In various embodiments, it can be desirable to use a starch with a particle size of no more than 5 microns. For example, in various embodiments as otherwise described herein, the starch is an amaranth starch. In other embodiments as otherwise described herein, the starch is a quinoa starch. In other embodiments as otherwise described herein, the starch is a rice starch. However, starches of larger granule size can be used, if the starch is fragmented to provide a desired median primary particle size. In other embodiments as otherwise described herein, the starch comprises a fragmented starch, e.g., a fragmented corn starch. Even starches of smaller granule size (e.g., amaranth, quinoa and rice) can be fragmented, for example, using mechanical force or hydrostatic pressure, to provide even smaller primary particles. Jet milling is one way to provide fragmented starches. And a variety of other starches can be used. Additionally, the starch may be a mixture of types of starch.

As used herein, “primary particles” of a starch are the smallest particles provided by the starch when it is dispersed in aqueous media. This will be granules or fragments thereof. Starches will typically be agglomerated into secondary particles made up of many primary particles (i.e., granules or fragments thereof), in order to provide a more convenient particle size for sale and use. In various embodiments as otherwise described herein, the starch has a median primary particle size in the range of 0.2-2 microns, e.g., 0.2-1 micron. In various embodiments as otherwise described herein, the starch has a median particle size in the range of 0.3-5 microns, e.g., 0.3-2 microns, or 0.3-1 microns. In various embodiments as otherwise described herein, the starch has a median particle size in the range of 0.4-5 microns, e.g., 0.4-2 microns, or 0.4-1 microns. In various embodiments as otherwise described herein, the starch has a median particle size in the range of 0.5-5 microns, e.g., 0.5-2 microns, 0.5-1 microns, or 1-5 microns. The starches desirably have the bulk of their primary particles within these size ranges. For example, in various such embodiments, at least 50%, e.g., at least 75%, or even at least 90%, of the primary particles of the inhibited starches have particle sizes in the range of 0.2-5 microns, e.g., 0.2-2 microns, or 0.2-1 micron. In various such embodiments, at least 50%, e.g., at least 75%, or even at least 90%, of the primary particles of the inhibited starches have particle sizes in the range of 0.3-5 microns, e.g., 0.3-2 microns, or 0.3-1 microns. In various such embodiments, at least 50%, e.g., at least 75%, or even at least 90%, of the primary particles of the inhibited starches have particle sizes in the range of 0.4-5 microns, e.g., 0.4-2 microns, or 0.4-1 microns. In various such embodiments, at least 50%, e.g., at least 75%, or even at least 90%, of the primary particles of the inhibited starches have particle sizes in the range of 0.5-5 microns, e.g., 0.5-2 microns, or 0.5-1 micron, or 1-5 microns. Light scattering is used to characterize median primary particle size of particles not in emulsions, using instruments well known in the field such as a Beckman Coulter LS 13320 laser diffraction Particle Size Analyzer or a Brookhaven Nano Brook DLS instrument. The starch can be dispersed in pH 6.5 RVA buffer containing an additional 1% NaCl to break up any agglomerates to ensure that the median size measurement is of dispersed primary particles.

Optical microscopy can also be used to assess the primary particles, especially when particles are stabilizing an emulsion. Starch materials can be stained with an iodine solution to improve visibility. Typically, the starch is visualized under bright field with or without polarized light. To prepare the starch, a 5% starch paste (which can be cooked or not) in RVA buffer with 1% NaCl at pH 6.5 is diluted with an equal volume of the same buffer, and then mixed with a further volume of 0.02N iodine solution. A drop of this mixture is added to a standard microscope slide and covered with a cover slip. The magnification is often 200×, but can take a range of values as required.

More detailed images of starches can be acquired with a scanning electron microscope (SEM). Backscattered imaging mode and low vacuum (40 Pa) are most typically used. A typical procedure is as follows: a small amount of sample powder is put on the surface of a double sided adhesive pad mounted on a specimen stub. A dust remover (e.g., Super Friendly AIR'IT™, FisherBrand) is used to blow away excess powder particles. The electron microscopy images are collected at 500× and 1500× magnification, although a range of magnification values can be used as required.

Optical microscopy and SEM observation of the starches of the disclosure indicated small median primary particle size in the low micron and sub-micron sizescale, in-line with the particle size commonly seen in Pickering emulsions.

In other embodiments, the inhibited starch having the protein content in the range of 0.2-8 wt % is of a larger particle size. Such starches can be fragmented when combined with ingredients for emulsification or foaming.

The present inventors have determined that inhibition can advantageously provide a starch that can stabilize an emulsion even in cooked systems, like various food and beverage products. Inhibited starches better retain their granular structure and resist fragmentation and dissolution when heat-processed in aqueous media, as is common in food and beverage production, e.g., through retorting or other cooking processes. Inhibited starches can vary with respect to their degree-of-inhibition, as characterized by their observed microscopy and sedimentation volume. Degree-of-inhibition can qualitatively be assessed by cooking the starch in water (typically cook at 95° C. for, e.g., 30 minutes with hand stirring in the first 6 minutes) and then observing the cooked starch under a microscope. Starches that have not been inhibited will have few granules and fragments, as they tend to dissolve in water during cooking. Starches that have been inhibited will show swollen intact particles under microscope. Highly inhibited starches exhibiting small and dark particles and starches that have been slightly inhibited exhibit large and light particles.

Degree-of-inhibition can be more quantitatively assessed through the measurement of the sedimentation volume of the starch. In various embodiments as otherwise described herein, the starches of the disclosure have a sedimentation volume in the range of 5-50 mL/g. For example, in various embodiments as otherwise described herein, the sedimentation volume is in the range of 10-50 mL/g, e.g., 15-50 mL/g, or 20-50 mL/g, or 25-50 mL/g. In various embodiments as otherwise described herein, the sedimentation volume is in the range of 5-40 mL/g, 10-40 mL/g, or 15-40 mL/g, or 20-40 mL/g, or 25-40 mL/g. In various embodiments as otherwise described herein, the sedimentation volume is in the range of 5-35 mL/g, 10-35 mL/g, or 15-35 mL/g, or 20-35 mL/g, or 25-35 mL/g.

As used herein, sedimentation volume is the volume occupied by one gram of cooked starch (dry basis) in 100 grams (i.e. total, including the starch) of salted buffer solution. This value is also known in the art as “swelling volume.” As used herein, the “salted buffer solution” refers to a solution prepared according to the following steps:

Using a top loader balance, weigh out 20 grams of sodium chloride into a 2-liter volumetric flask containing a stir bar;

To this add RVA pH 6.5 buffer (purchased from Ricca Chemical Company) so that the flask is at least half full;

Stir to mix until sodium chloride is dissolved;

Add additional RVA pH 6.5 buffer to a final volume of 2 liters;

Sedimentation volumes as described herein are determined by first cooking the starch at 5% solids in the salted buffer solution by suspending a container containing the slurry in a 95° C. water bath and stirring with a glass rod or metal spatula for 6 minutes, then covering the container and allowing the paste to remain at 95° C. for an additional 20 minutes. The container is removed from the bath and allowed to cool on the bench.

The resulting paste is brought back to the initial weight by addition of water (i.e., to replace any evaporated water) and mixed well. 20.0 g of the paste (which contains 1.0 g starch) is weighted into a 100 mL graduated cylinder containing salted buffer solution, and the total weight of the mixture in the cylinder is brought to 100 g using the buffer. The cylinder is allowed to sit undisturbed for 24 hours. The volume occupied by the starch sediment (i.e., as read in the cylinder) is the sedimentation volume for 1 g of starch, i.e., in units of mL/g.

Sedimentation volumes of fragmented starches are measured on a corresponding unfragmented starch, i.e., a starch that is prepared identically to the fragmented starch, but without fragmentation. Thus, when the starch is a fragmented starch, e.g., a fragmented corn starch, it can be inhibited using conditions that provide a sedimentation volume in the range of 5-50 mL/g to a corresponding un-fragmented starch. In various embodiments, the fragmented starch is inhibited using conditions that provide a sedimentation volume in the range of 10-50 mL/g, e.g., 15-50 mL/g, or 20-50 mL/g, or 25-50 mL/g, to a corresponding un-fragmented starch. In various embodiments, the fragmented starch is inhibited using conditions that provide a sedimentation volume in the range of 5-40 mL/g, 10-40 mL/g, or 15-40 mL/g, or 20-40 mL/g, or 25-40 mL/g, to a corresponding un-fragmented starch. In various embodiments, the fragmented starch is inhibited using conditions that provide a sedimentation volume in the range of 5-35 mL/g, 10-35 mL/g, or 15-35 mL/g, or 20-35 mL/g, or 25-35 mL/g, to a corresponding un-fragmented starch.

The present inventors have determined that a relatively high amount of protein is desirably present in inhibited starches used in stabilization of emulsions and foams. Without intending to be bound by theory, it is believed that the protein provides a degree of hydrophobicity to the starch primary particles, which improves adsorption at water/oil and water/air interfaces. Accordingly, the starches of the disclosure have a protein content in the range of 0.2 to 8 wt % protein on a dry starch basis. For example, in various embodiments as otherwise described herein, the inhibited starch has a protein content in the range of 0.2 to 5 wt % protein, e.g., in the range of 0.2 to 3 wt % on a dry starch basis. In various embodiments as otherwise described herein, the inhibited starch has a protein content in the range of 0.3 to 8 wt % protein, e.g., in the range of 0.3 to 5 wt %, or 0.3 to 3 wt % on a dry starch basis. In various embodiments as otherwise described herein, the inhibited starch has a protein content in the range of 0.4 to 8 wt % protein, e.g., in the range of 0.4 to 5 wt %, or 0.4 to 3 wt % on a dry starch basis. In various embodiments as otherwise described herein, the inhibited starch has a protein content in the range of 0.5 to 8 wt % protein, e.g., in the range of 0.5 to 5 wt %, or 0.5 to 3 wt % on a dry starch basis. In various embodiments as otherwise described herein, the inhibited starch has a protein content in the range of 0.6-8 wt % protein, e.g., in the range of 0.6-5 wt %, or 0.6-3 wt %, or 0.7-8 wt %, or 0.7-5 wt %, or 0.7-3 wt % on a dry starch basis. In various embodiments as otherwise described herein, the inhibited starch has a protein content in the range of 1 to 8 wt % protein, e.g., in the range of 1 to 5 wt %, or 1 to 3 wt % on a dry starch basis. The person of ordinary skill in the art can determine protein content using the Dumas method as described in the Examples below.

The inhibited starches of the present disclosure can have a variety of viscosities as measured by a Rapid Visco Analyzer (RVA). For example, in various embodiments the inhibited starch as otherwise described herein can have a viscosity as measured by RVA is in the range of 50-1500 cP at 5% solids. In various such embodiments, the viscosity as measured by RVA at 5% solids is in the range of 50-1000 cP, 50-850 cP, 50-700 cP, 50-500 cP, 50-400 cP, 50-300 cP, 50-200 cP, 100-1100 cP, 100-1000 cP, 100-850 cP, 100-700 cP, 100-500 cP, 100-400 cP, 100-300 cP, 200-1100 cP, 200-1000 cP, 200-850 cP, 200-700 cP, 200-500 cP, 400-1100 cP, 400-1000 cP, 400-850 cP, 400-700 cP, 600-1100 cP, 600-850 cP, 700-1500 cP, or 700-1300 cP.

The viscosity is measured by RVA at 5% solids in a pH 6.5 phosphate buffer at 1% NaCl at a stir rate of 160 rpm. The initial temperature of the analysis is 50° C.; the temperature is ramped linearly up to 90° C. over 3 minutes, then held at 95° C. for 20 minutes, then ramped linearly down to 50° C. over 3 minutes, then held at 50° C. for 9 minutes, after which time the viscosity is measured. Notably, when a pasting peak is displayed at times of about 2-5 minutes, the final viscosity measured is higher than the pasting peak viscosity. When the pasting peak is absent, the viscosity during the 95° C. hold is flat, or increases.

The starches of the disclosure can be inhibited using a variety of techniques. For example, chemical inhibition is a conventional inhibition technique in which chemical crosslinking agents are used to crosslink the starch to provide inhibition. In various embodiments as otherwise described herein, the inhibited starch is chemically inhibited, e.g., by crosslinking with phosphate, adipate, epichlorohydrin or acrolein. Reagents and process conditions for such crosslinking are familiar to the person of ordinary skill in the art.

Notably, however, the inhibited starches described herein can be made without many of the conventional chemical reagents used in the making of conventional chemically-modified and/or inhibited starches. Accordingly, in various embodiments, the starches as otherwise described herein can be marked or labeled as so-called “clean-label” starches.

Thus, in various desirable embodiments, the starch is not chemically inhibited. In various embodiments, the inhibited starch product is not crosslinked with phosphate. In various embodiments, the inhibited starch product is not crosslinked with adipate. In various embodiments, the inhibited starch product is not crosslinked with epichlorohydrin. In various embodiments, the inhibited starch product is not crosslinked with acrolein.

In various desirable embodiments, the starch is thermally inhibited. As the person of ordinary skill in the art will appreciate, there are a variety of thermal inhibition processes that involve heating the starch, for example, under dry conditions at high temperatures. Often the starch is subjected to a pH adjustment in conjunction with the heating. A variety of thermal inhibition processes are described in more detail below.

Similarly, a variety of chemical modifications are known to adjust properties of starches. Accordingly, in various embodiments, a starch of the disclosure is chemically modified, e.g., one or more of succinated (e.g., octenylsuccinated), acetated, adipated, hydroxyethylated, hydroxypropylated, carboxymethylated, or oxidized.

However, as described above, the present inventors have noted that chemical modification is not necessary to provide advantageous materials, and that in many circumstances it can be desirable to provide a clean-label starch. Thus, it can be desirable to provide starches that lack certain chemical modifications. For example, in various embodiments as otherwise described herein, the inhibited starch is not acetylated. In various embodiments as otherwise described herein, the inhibited starch is not adipated. In various embodiments as otherwise described herein, the inhibited starch is not hydroxypropylated. In various embodiments as otherwise described herein, the inhibited starch is not hydroxyethylated. In various embodiments as otherwise described herein, the inhibited starch is not carboxymethylated. In various embodiments as otherwise described herein, inhibited starch is not phosphated. In various embodiments as otherwise described herein, the inhibited starch is not succinated (e.g., not octenylsuccinated). In various embodiments as otherwise described herein, the inhibited starch has substantially no fatty acid residues. In various embodiments as otherwise described herein, the inhibited starch is not cationic or zwitterionic. Thus, in various embodiments as otherwise described herein as otherwise described herein, the inhibited starch is not bleached or oxidized, e.g., with peroxide or hypochlorite.

The inhibited starches described herein can be made with relatively little color. For example, various embodiments of the inhibited starches as otherwise described herein are relatively low in color, i.e., have a Yellowness Index of no more than 10, for example, in the range of 3-10 or 5-10. In various desirable embodiments, the inhibited starches described herein are especially low in color, i.e., the Yellowness Index is less than 8 (e.g., 3-8 or 5-8). Yellowness Index is determined via ASTM E313.

As noted above, a variety of processes can be used to provide an inhibited starch, e.g., with a sedimentation volume in the range of 5 mL/g to 50 mL/g, or in any other range described herein.

Native starch can be obtained commercially, or can be isolated from a corresponding flour under low shear at alkaline conditions.

A variety of inhibition processes can be used to inhibit the starches as described herein. For example, in various embodiments as otherwise described herein, conventional chemical modification can be used to inhibit the starch by reaction with a crosslinking agent. Crosslinking agents suitable for this purpose include acrolein, phosphate (e.g., using POCl₃ or sodium trimetaphosphate), acetic adipic anhydride and epichlorohydrin. An example of a crosslinking process is using POCl₃ as crosslinking agent to provide a phosphate-crosslinked starch. The person of ordinary skill in the art can adapt conventional chemical modification processes for inhibiting the starches described herein.

In other embodiments, the starch is inhibited using a thermal process, for example, by adjusting the pH of the starch to neutral or greater (e.g., 8-9.5), then dehydrating the starch and heat treating it for a time and temperature sufficient to inhibit the starch, e.g., 120-180° C. for up to 20 hours). Such thermal processes for inhibition are familiar to the person of ordinary skill in the art.

In other embodiments as otherwise described herein, the starch is inhibited using a method as described in International Patent Application Publication no. WO 2013/173161, which is hereby incorporated herein by reference in its entirety. Thus, a method for inhibiting starch for use in the methods described herein can include (a) heating a non-pregelatinized starch in an alcoholic (e.g., ethanolic) medium in the presence of a base at a temperature of at least 35° C.; (b) neutralizing the base with an acid; (c) separating the inhibited starch from the alcoholic medium; and (d) removing alcohol solvent from the inhibited starch, e.g., by heating or with steam.

The alcoholic medium generally comprises at least one alcohol, particularly a C₁-C₄ monoalcohol such as methanol, ethanol, n-propanol, isopropanol, n-butanol, t-butyl alcohol and the like. One or more other substances may also be present in the alcoholic medium, such as a non-alcoholic organic solvent (particularly those that are miscible with the alcohol) and/or water. However, in one embodiment of the method the alcoholic medium does not contain any solvent other than alcohol and, optionally, water. Aqueous alcohols, for example, may be used to advantage. The alcoholic medium may comprise, for instance, 30% to 100% by weight alcohol (e.g., ethanol) and from 0% to 70% by weight water. In one embodiment, the alcoholic medium contains from 80% to 96% by weight alcohol (e.g., ethanol) and from 4% to 20% by weight water, the total amount of alcohol and water equaling 100%. In another embodiment, the alcoholic medium contains 90% to 100% by weight alcohol (e.g., ethanol) and from 0% to 10% by weight water, the total amount of alcohol and water equaling 100%. In other embodiments, not more than 10% or not more than 15% by weight water is present in the alcoholic medium. The quantity of alcoholic medium relative to starch is not considered to be critical, but typically for the sake of convenience and ease of processing sufficient alcoholic medium is present to provide a stirrable and/or pumpable slurry. For example, the weight ratio of starch:alcoholic medium may be from about 1:2 to about 1:6.

In various methods, at least some amount of treatment agent (e.g., base and/or salt) is present when the starch is heated in the alcoholic medium. However, it is advantageous that large amounts of treatment agent (relative to starch) need not be used in order to achieve effective inhibition of the starch, in contrast to previously known starch modification processes. This simplifies the subsequent processing of the inhibited starch and lowers potential production costs. Typically, at least 0.5% by weight of treatment agent (based on the dry weight of starch used) is employed, although in other embodiments at least 1%, at least 2%, at least 3%, at least 4% or at least 5% by weight of treatment agent is present. For economic reasons, generally no more than 10% or 15% by weight of treatment agent is present.

Typically, the mixture of starch, alcoholic medium and treatment agent is in the form of a slurry. In various embodiments, it may be desirable to adjust the pH of the slurry to a particular value. It can be difficult to measure the pH of such a slurry due to the presence of the alcohol. In an embodiment where it is desired to make the slurry basic by adding a base, a suitable amount of base can be determined as if the slurry is a slurry of starch in de-ionized water alone and then scaled up to the actual amount while keeping the same ratio of base and starch.

The slurry may, for example, be neutral (pH 6 to 8) or basic (pH greater than 8). In one embodiment, the pH of the slurry is at least 6. In another embodiment, the pH of the slurry is at least 7. The slurry pH in another embodiment is not more than 12. In other embodiments, the pH of the slurry is 6-10, 7.5-10.5 or 8-10. In still other embodiments, the pH of the slurry is 5-8 or 6-7.

The treatment of the starch may be effected by first placing the starch in the alcoholic medium and then adding treatment agent (e.g., base and/or salt). Alternatively, the treatment agent may be first combined with the alcoholic medium and then contacted with the starch. The treatment agent may be formed in situ, such as by separately adding a base and an acid which react to form the salt which functions as the treatment agent.

Suitable bases for use in the process include, but are not limited to, alkali metal and alkaline earth metal hydroxides such as potassium hydroxide, calcium hydroxide and sodium hydroxide.

Suitable salts for use in these methods include water-soluble substances that ionize in aqueous solution to provide a substantially neutral solution (i.e., a solution having a pH of from 6 to 8). Alkali metal-containing salts are particularly useful, as are salts of organic acids (e.g., a sodium or potassium salt) such as itaconic acid, malonic acid, lactic acid, tartaric acid, citric acid, oxalic acid, fumaric acid, aconitic acid, succinic acid, oxalosuccinic acid, glutaric acid, ketoglutaric acid, malic acid, fatty acids and combinations thereof.

Mixtures of different treatment agents may be used. For example, the starch may be heated in the alcoholic medium in the presence of both at least one base and at least one salt.

The starch, alcoholic medium and treatment agent are heated for a time and at a temperature effective to inhibit the starch to the desired extent. Generally speaking, temperatures in excess of room temperature (i.e., 35° C. or greater) will be necessary. At the same time, extremely high temperatures should be avoided. The heating temperature can be, for example, 35° C. to 200° C. Typically, temperatures of from 100° C. to 190° C., 120° C. to 180° C., or from 130° C. to 160° C., or from 140° C. to 150° C. will be sufficient. The heating time generally is at least 5 minutes but no more than 20 hours and typically 40 minutes to 2 hours. In general, a desired level of starch inhibition may be achieved more rapidly if the heating temperature is increased.

When the temperature selected for the heating step exceeds the boiling point of one or more components of the alcoholic medium, it will be advantageous to carry out the heating step in a vessel or other apparatus capable of being pressurized. The treatment may be conducted within a confined zone in order to maintain the alcoholic medium in a liquid state. Additional positive pressure could be employed, but is generally not necessary. The starch may be slurried in the alcoholic medium together with the treatment agent under conditions of elevated temperature and pressure and treated for a time sufficient to change the starch's viscosity characteristics. Such treatment may be conducted in a stirred tank reactor on a batch basis or in a tubular reactor on a continuous basis, although other suitable processing techniques will be apparent to those skilled in the art. In another embodiment, the starch may be in the form of a bed within a tubular reactor and a mixture of the alcoholic medium and treatment agent passed through such bed (optionally, on a continuous basis), with the bed being maintained at the desired temperature to effect inhibition of the starch.

In embodiments in which a base has been utilized as a treatment agent, the mixture of starch, alcoholic medium and base may be combined with one or more acids, once the heating step is completed, for the purpose of neutralizing the base. Suitable acids for use in such neutralization step include, but are not limited to, carboxylic acids such as itaconic acid, malonic acid, lactic acid, tartaric acid, oxalic acid, fumaric acid, aconitic acid, succinic acid, oxalosuccinic acid, glutaric acid, ketoglutaric acid, malic acid, citric acid, fatty acids and combinations thereof, as well as other types of acids such as uric acid. If the inhibited starch is intended for use as a food ingredient, the acid generally should be selected to be one that is permitted for such use under applicable regulations. Typically, sufficient acid is added to lower the pH of the mixture to about neutral to slightly acidic, e.g., a pH of from about 5 to about 7 or from about 6 to about 6.5.

The neutralization with acid may be carried out at any suitable temperature. In one embodiment, the slurry of starch, base and alcoholic medium is cooled from the heating temperature used to approximately room temperature (e.g., about 15° C. to about 30° C.) prior to being combined with the acid to be used for neutralization. The neutralized mixture may thereafter be further processed as described below to separate the inhibited starch from the alcoholic medium. In another embodiment, however, neutralization of the base is followed by further heating of the starch slurry. Such further heating has been found to be capable of modifying the rheological properties of the inhibited starch obtained, as compared to the viscosity characteristics of an analogously prepared starch that has not been subjected to heating after neutralization of the base.

Generally speaking, such further heating step is advantageously carried out at temperatures in excess of room temperature (i.e., 35° C. or greater). At the same time, extremely high temperatures should be avoided. The heating temperature can be, for example, 35° ° C. to 200° C. Typically, temperatures of from 100° C. to 190° C., 120° C. to 180° C., or from 130° ° C. to 160° C., or from 140° C. to 150° C. will be sufficient. The heating time generally is at least 5 minutes but no more than 20 hours and typically 40 minutes to 2 hours.

The mixture of starch and alcoholic medium may be processed so as to separate the starch from the alcoholic medium. Conventional methods for recovering particulate solids from liquids such as filtration, decantation, sedimentation or centrifugation may be adapted for such purpose. The separated starch may optionally be washed with additional alcoholic medium and/or alcohol and/or water to remove any undesired soluble impurities. In one embodiment, neutralization of residual base is accomplished by washing the recovered starch with an acidified liquid medium. Drying of the separated starch will provide an inhibited non-pregelatinized starch in accordance with the disclosure. For example, drying may be performed at a moderately elevated temperature (e.g., 30° C. to 60° C.) in a suitable apparatus such as an oven or a fluidized bed reactor or drier or mixer. Vacuum and/or a gas purge (e.g., a nitrogen sweep) may be applied to facilitate removal of volatile substances (e.g., water, alcohol) from the starch. The resulting dried inhibited non-pregelatinized starch may be crushed, ground, milled, screened, sieved or subjected to any other such technique to attain a particular desired particle size. In one embodiment, the inhibited starch is in the form of a free-flowing material.

In one embodiment, however, the starch is subjected to a desolventization step at a significantly higher temperature (e.g., greater than 80° C. or greater than 100° C. or greater than 120° C.). Excessively high temperatures should be avoided, however, since degradation or discoloration of the starch may result. Such a step not only reduces the amount of residual solvent (alcohol) in the product but also provides the additional unexpected benefit of enhancing the degree of inhibition exhibited by the starch. Desolventization temperatures can, for example, be about 100° C. to about 200° C. Typical temperatures are 120° C. to 180° C. or 150° C. to 170° C. The desolventization may be carried out in the presence or in the absence of steam. Steam treatment has been found to be advantageous in that it helps to minimize the extent of starch discoloration which may otherwise occur at such an elevated temperature. In one embodiment, steam is passed through a bed or cake of the inhibited starch. The starch desolventization methods of U.S. Pat. No. 3,578,498, incorporated herein by reference in its entirety for all purposes, may be adapted for use. Following steam treatment, the inhibited starch may be dried to reduce the residual moisture content (e.g., by heating in an oven at a temperature of from about 30° C. to about 70° C. or in a fluidized bed reactor).

In one embodiment, the treated starch, which has been recovered from the alcoholic medium, is first brought to a total volatiles content of not more than about 35% by weight or not more than about 15% by weight. This can be accomplished, for example, by first air or oven drying the recovered starch at moderate temperature (e.g., 20° C. to 70° C.) to the desired initial volatiles content. Live steam is then passed through the dried starch, the system being maintained at a temperature above the condensation point of the steam. A fluid bed apparatus may be used to perform such a steam desolventization step.

In general, it will be desirable to carry out desolventization under conditions effective to result in a residual alcohol content in the inhibited starch of less than 1 weight % or less than 0.5 weight % or less than 0.1 weight %.

Following desolventization, the inhibited starch may be washed with water and then re-dried to further improve color and/or flavor and/or reduce the moisture content.

Of course, the person of ordinary skill in the art can use other methodologies to inhibit the starches described herein. The starch can, for example, be subjected to a pH adjustment and then heated. The pH adjustment can be performed by contacting a pH-adjusting agent with the starch; examples of pH-adjusting agents include acids (e.g., an organic acid or and inorganic acid). Examples of acids that may be suitable for use according to the present disclosure include sulfuric acid, phosphoric acid, hydrochloric acid, itaconic acid, aconitic acid, malonic acid, lactic acid, tartaric acid, oxalic acid, fumaric acid, aconitic acid, succinic acid, acetic acid, oxalosuccinic acid, glutaric acid, ketoglutaric acid, malic acid, citric acid, fatty acids and carbonic acid, as well as salts thereof (e.g., potassium and/or sodium salts, which can be generated in situ by neutralization of the acid) and combinations thereof. The pH-adjusting agent can be contacted with the starch in any convenient fashion, e.g., as a slurry in liquid (e.g., water, alcohol (e.g., as described above, including ethanol or isopropanol), including aqueous alcohol such as aqueous ethanol, or another solvent); in dry form; in damp form (e.g., in a mist in a solvent (such as water, aqueous ethanol, or another solvent); or in the form of a damp dough of the starch (e.g., with water, aqueous ethanol, or another solvent). And when an alkali metal salt of an acid is to be used, it can be formed in situ, e.g., by adding the acid and an alkali metal hydroxide or carbonate in separate steps.

The pH adjustment can be performed to yield a variety of pH values. For example, in various embodiments, and as described in WO 2013/173161, the pH adjustment can be performed to yield a pH in the range of 7-10. In other, alternative embodiments, the pH adjustment can be performed to yield a pH in the range of 2-7, e.g., in the range of 2-6, or 2-5, or 2-4, or 2-3, or 3-7, or 3-6, or 3-5, or 3-4, or 4-7, or 4-6, or 4.5-7, or 4.5-6, or 5-7, or 5-6, or about 2.5, or about 3, or about 3.5, or about 4, or about 4.5, or about 5, or about 5.5, or about 6, or about 6.5, or about 7. When the pH adjustment is performed in a slurry, the pH of the slurry is the relevant pH. When the pH adjustment is performed in a substantially non-liquid form (e.g., a dough, or in damp solid), the pH of the solid material at 38% in water is the relevant pH. The amount of the pH-adjusting agent relative to the starch can vary, for example, from 0.05-30 wt % on a dry solids basis, e.g., 0.05-20 wt %, 0.05-10 wt %, 0.05-5 wt %, 0.05-2 wt %, 0.05-1 wt %, 0.05-0.5 wt %, 0.2-30 wt %, 0.2-20 wt %, 0.2-10 wt %, 0.2-5 wt %, 0.2-2 wt %, 0.2-1 wt %, 1-30 wt %, 1-20 wt %, 1-10 wt %, 1-5 wt %, 5-30 wt % or 5-20 wt %. Desirably, the pH adjusting agent is mixed thoroughly with the starch feedstock. This will require different process conditions depending on the form in which the pH adjustment is performed. If the pH adjustment is performed in a slurry, simply stirring the slurry for a few minutes may be sufficient. If the pH adjustment is performed in a drier form (e.g., in a damp solid or a dough), more substantial contacting procedures may be desirable. For example, if a solution of the pH-adjusting agent is sprayed onto dry starch feedstock, it can be desirable to mix for about 30 minutes then store for at least a few hours. It is desirable to provide for uniform distribution of the pH-adjusting agent throughout the starch, i.e., on a granular level, in order to provide uniform inhibition.

After the pH-adjusting agent is contacted with the starch, the starch can be heated (i.e. while still in contact with pH-adjusting agent). The starch can be heated in a variety of forms. For example, the starch can be heated in alcohol or non-aqueous solvent slurry (e.g., under pressure if the boiling point of the solvent not sufficiently above the heating temperature); as a dough of starch, water, and non-water solvent to suppress granular swelling (e.g., as disclosed in WO 2013/173161), or in a substantially dry state, e.g., at a moisture level of less than 5%, less than 4%, or less than 3% (solvent can be removed using conventional techniques such as filtration, centrifugation and/or heat-drying, e.g. as described above with respect WO 2013/173161). The starch can be, for example, dried to a moisture level of less than 5% before further heating, in order to suppress gelatinization of the starch. Relatively low temperatures, e.g., 40-80° C., or 40-60° C., or about 50° C., can be used for such drying. Vacuum can also be used in the drying process. The starch can be dried as a result of the heating process (see below); a separate drying step is not necessary.

The dried starch can be heated at a variety of temperatures for a variety of times in order to inhibit it to a desired degree. One suitable temperature range is the range of 100-200° C. For example, in various methods, the heating temperature is 120-160° C. In other various methods, the heating temperature is 120-200° C., 120-180° C., or 120-160° C., or 120-140° C., or 140-200° C., or 140-180° C., or 140-160° C., or 160-200° C., or 160-180° C., or 180-200° C. The starch can be heated for a time in the range of, for example, 20 seconds to 20 hours. Typical heating times are in the range of 10 minutes to two hours. Longer heating times and/or higher heat-treatment temperatures can be used to provide more inhibition. The material is desirably uniformly heated. As examples, the starch can be heated under pressure to maintain a desired moisture content, or it can be heated in a mass flow bin or similar device.

Various methods described herein can be practiced, for example, using no alcohol in the liquid medium for the contacting with the pH adjustment. In various particularly desirable methods, water is used as the medium for the pH adjustment. Accordingly, in various desirable embodiments, the inhibited starch comprises less than 500 ppm of alcohol solvent, e.g., less than 500 ppm ethanol. For example, in various embodiments, the inhibited starch comprises less than 100 ppm, less than 50 ppm, less than 10 ppm, less than 5 ppm, or less than 1 ppm of alcohol solvent, e.g., less than 100 ppm, less than 50 ppm, less than 10 ppm, less than 5 ppm, or less than 1 ppm ethanol.

The heated starch can be allowed to cool then used as-is, or further treated as is conventional in the art. For example, the starch can be washed to provide even whiter color and more pleasant flavor. If a non-aqueous solvent is used, it can be desirable to remove as much solvent as possible. But if relatively low levels of the pH-adjusting agent are used, the final product can meet reasonable pH and ash targets without further washing.

The heat treatments described herein are desirably performed to avoid substantially gelatinizing the starch during inhibition. Thus, the specific conditions of time of treatment, temperature of treatment, and proportions of the components of the mixture of starch, solvent and treatment agent are generally selected such that the starch is not gelatinized to a significant extent. That is, the starch remains substantially non-pregelatinized during the inhibition process as described above. In some embodiments, however, the inhibited starch is preferably gelatinized.

In various embodiments as otherwise described herein, the starch product is not crosslinked by acrolein, phosphate, adipate or epichlorohydrin, i.e., the starch product is inhibited via a heat treatment. In various other embodiments, the starch product is chemically inhibited by crosslinking with phosphate, adipate, acrolein or epichlorohydrin.

While it can be preferable, in various embodiments, for the starches of the disclosure to not be chemically modified, in various other embodiments chemical modification of the starches can be useful to further modify starch properties. Such starches can be chemically modified, for example, by ether substitution (e.g., ethyl, hydroxypropyl) or ester substitution (e.g., acetate, octenyl succinic anhydride).

As the person of ordinary skill in the art will appreciate, the starch may be purified, e.g., by conventional methods, to reduce undesirable flavors, odors, or colors, e.g., that are native to the starch or are otherwise present. For example, methods such as washing (e.g., alkali washing), steam stripping, ion exchange processes, dialysis, filtration, bleaching such as by chlorites, and/or centrifugation can be used to reduce impurities. The person of ordinary skill in the art will appreciate that such purification operations may be performed at a variety of appropriate points in the process. However, such purification processes should be performed so as to retain a desired protein content, as the present inventors have determined that some degree of protein should be present in order to provide the starch with a desired degree of hydrophobicity for emulsion and foam stability.

The starches of the disclosure can be provided in uncooked form (i.e., not gelatinized). Such starches can, for example, be gelatinized during processing to provide emulsions and foams as described herein. In other embodiments, the starches can be provided in cooked form (i.e., gelatinized). Such starches can be provided as a product in pregelatinized form, such that cooking is not necessary to provide a gelatinized starch in an emulsion or foam. And as described above, gelatinization can occur during processing, e.g., to provide an emulsion or foam including the gelatinized starch. The person of ordinary skill in the art can use polarized microscopy to determine whether a starch is gelatinized or ungelatinized.

The present inventors have surprisingly determined that the starches of the disclosure can, in cooked form, stabilize emulsions and foams. Accordingly, the present disclosure provides for stable emulsions and foams even in cooked systems, i.e., as typically provided in various food and beverage products.

The present inventors contemplate emulsion systems that can be stabilized (as quantified, e.g., by emulsion (or creaming) index) using the gelatinized starches described herein, e.g., as an oil-in-water emulsion. Thus, another aspect of the disclosure is an emulsion comprising an emulsified phase that is a hydrophobic phase, emulsified within a hydrophilic phase, stabilized by a gelatinized starch as described herein. Another aspect of the disclosure is an emulsion comprising an emulsified phase that is a hydrophobic phase emulsified within a hydrophilic phase, stabilized by a gelatinized inhibited starch, wherein the gelatinized inhibited starch has a median primary particle size in the range of 0.2-5 microns and a protein content in the range of 0.2-8 wt. %. In various desirable embodiments, the hydrophilic phase is an aqueous phase, and the hydrophobic phase is an oil or fat phase.

The primary particles of the gelatinized starch as described herein can stabilize the emulsion by adsorbing at the hydrophobic/hydrophilic interface, as do small particles in so-called Pickering emulsions. Notably, the starches of the disclosure can retain emulsifying capability even when cooked and sheared during processing, as is the typical case in the preparation of many food and beverage products. The median primary particle size of the starch may decrease under shear in processing during emulsification. However, starches of the disclosure can be provided that remain within the described size ranges even after processing. It is noted that the starch as introduced into the emulsification process may have a primary particle size within the described ranges, or can be of larger primary particle size and emulsified under conditions that form the starch having the primary particle sizes as described herein.

The amount of starch used in the emulsion can vary, but can be at generally low amounts. In various embodiments as otherwise described herein, the starch is present in an amount in the range of at least 0.5 wt % of the emulsified phase, e.g., in the range of 0.5-15 wt %, e.g., 0.5-10 wt %, or 0.5-7 wt %, or 1-15 wt %, or 1-10 wt % of the emulsified phase, or 1-7 wt %. Notably, in various embodiments, especially amounts of starch can be used, e.g., in the range of 0.5-5 wt %, or 1-5 wt % of the emulsified phase. The person of ordinary skill in the art will determine a desired use rate of the starch based on the disclosure herein.

Emulsions can be made with a variety of droplet sizes, depending, e.g., on the identities and the relative amounts of the starch and the emulsified phase, and the conditions used to emulsify. For example, in various embodiments as otherwise described herein, the emulsion has a median emulsion droplet size (i.e., of the emulsified phase) in the range of 0.5-100 microns. For example, in various embodiments as otherwise described herein, the emulsion has a median emulsion droplet size in the range of 0.5-75 microns, or 0.5-50 microns, or 0.5-25 microns, or 0.5-15 microns, or 1-100 microns, or 1-50 microns, or 1-25 microns, or 1-15 microns. In various embodiments as otherwise described herein, the emulsion has a median emulsion droplet size in the range of 2-100 microns, or 2-50 microns, or 2-25 microns, or 2-15 microns.

As noted above, the starches of the disclosure can be useful in stabilizing foams, i.e., with gas bubbles stabilized within a liquid phase by starch particles adsorbed at the air/liquid interface. Accordingly, another aspect of the disclosure is a foam comprising gas bubbles stabilized within a liquid phase, stabilized by a gelatinized starch as described herein. Another aspect of the disclosure is a foam comprising gas bubbles stabilized within a liquid phase, stabilized by a gelatinized inhibited starch, wherein the gelatinized inhibited starch has a median primary particle size in the range of 0.2-5 microns and a protein content in the range of 0.2-8 wt. %. The foam can have a variety of bubble sizes, depending, e.g., on processing conditions and the identities of the liquid phase and the starch. For example, in various embodiments, the foam has a median bubble size in the range of 20-3000 microns, e.g., 20-1000 microns, or 20-500 microns, or 20-100 microns.

In various embodiments, the gelatinized inhibited starch of the emulsion or foam is a rice starch, an amaranth starch, a quinoa starch or a rice starch.

In various embodiments, the gelatinized inhibited starch of the emulsion or foam is a gelatinized form of an inhibited unfragmented starch that has a sedimentation volume of 5-50 mL/g. The sedimentation volume can have any value as described above for inhibited unfragmented starches.

In various embodiments, the gelatinized inhibited starch of the emulsion or foam is a fragmented starch, e.g., a fragmented corn starch. The gelatinized inhibited starch can, in various embodiments, be inhibited using conditions that provide a sedimentation volume in the range of 5-50 mL/g to a corresponding un-fragmented starch. The inhibition can be performed on unfragmented starch, then the fragmentation can be later (e.g., in situ during emulsion/foam preparation) performed to provide the inhibited fragmented starch. The sedimentation volume can have any value as described above for inhibited fragmented starches.

In various embodiments, the gelatinized inhibited starch of the emulsion or foam has a median primary particle size in the range of 0.2-2 microns, e.g., 0.2-1 micron. In various embodiments, the gelatinized inhibited starch of the emulsion or foam has a median particle size in the range of 0.5-5 microns, e.g., 0.5-2 microns. or 1-5 microns. Particle sizes in emulsions can be determined by microscopy.

In various embodiments, at least 50%, e.g., at least 75%, or even at least 90%, of the primary particles of the gelatinized inhibited starch of the emulsion have particle sizes in the range of 0.2-5 microns, e.g., 0.2-2 microns, or 0.2-1 micron. In various embodiments, at least 50%, e.g., at least 75%, or even at least 90%, of the primary particles of the gelatinized inhibited starch of the emulsion or foam have particle sizes in the range of 0.5-5 microns, e.g., 0.5-2 microns, or 1-5 microns.

In various embodiments, the gelatinized inhibited starch of the emulsion or foam is the gelatinization product of a starch having a protein content in the range of 0.2-5 wt % protein, e.g., in the range of 0.2-3 wt % on a dry starch basis. In various embodiments, the gelatinized inhibited starch of the emulsion or foam is the gelatinization product of a starch having a protein content in the range of 0.3-8 wt % protein, e.g., in the range of 0.3-5 wt %, or 0.3-3 wt % on a dry starch basis. In various embodiments, the gelatinized inhibited starch of the emulsion or foam is the gelatinization product of a starch having a protein content in the range of 0.4-8 wt % protein, e.g., in the range of 0.4-5 wt %, or 0.4-3 wt % on a dry starch basis. In various embodiments, the gelatinized inhibited starch of the emulsion or foam is the gelatinization product of a starch having a protein content in the range of 0.5-8 wt % protein, e.g., in the range of 0.5-5 wt %, or 0.5-3 wt % on a dry starch basis. In various embodiments, the gelatinized inhibited starch of the emulsion or foam is the gelatinization product of a starch having a protein content in the range of 0.6-8 wt % protein, e.g., in the range of 0.6-5 wt %, or 0.6-3 wt %, or 0.7-8 wt %, or 0.7-5 wt %, or 0.7-3 wt % on a dry starch basis. In various embodiments, the gelatinized inhibited starch of the emulsion or foam is the gelatinization product of a starch having a protein content in the range of 1-8 wt % protein, e.g., in the range of 1-5 wt %, or 1-3 wt % on a dry starch basis.

The gelatinized inhibited starch of the emulsions or foams can, in various embodiments, can, be inhibited using any of the techniques described above, and can be chemically modified, or, in many embodiments, not modified, with any of the modifications described above.

As the person of ordinary skill in the art will appreciate, some food and beverage products can be in the form of both an emulsion and a foam. For example, desserts like ice cream and mousses can often include both emulsified fat and air bubbles.

The emulsions and foams described herein can find use in a wide variety of products. The present inventors have determined that the starches described herein can provide good stabilization of foams and emulsions without an undesirable effect on flavor. The starches can be provided with a desired tolerance to processing variables such as heat, shear and extremes of pH, particularly for a significant time under such conditions, and with a rheological and textural stability over a desired shelf-life.

For example, in various embodiments, an emulsion or foam as otherwise described herein is in the form of a food or beverage product. In various embodiments, the food or beverage product is a gravy, a sauce (e.g., a mayonnaise, a white sauce or a cheese sauce), a soup, or a stew. In various embodiments, the food or beverage product is a dressing such as a salad dressing (e.g., pourable or spoonable). In various embodiments, the food or beverage product is a dairy product, e.g. a yogurt, a sour cream, an ice cream or an ice milk. In various embodiments, the food or beverage product is a dairy substitute, such as a non-dairy creamer, a plant milk (such as an oat milk, a soy milk or a nut milk) or a food or beverage based thereon (e.g., an ice cream analog based on such milks), or a margarine. In various embodiments, the food or beverage product is a cream filling or a custard. In various embodiments, the food or beverage product is a confectionary, e.g., a chocolate. In various embodiments, the food or beverage product is a mousse, a smoothie or a shake. However, the person of ordinary skill in the art will appreciate that a wide variety of other food and beverage products can advantageously include emulsions and/or foams as described herein.

The emulsions and foams can also find use in personal care products. A wide variety of personal care products, many in the forms of lotions or creams, include emulsified systems and/or foams. Examples include shaving creams, skin lotions, hair conditioners, hair products, e.g., in the form of mousses and gels, sunscreens, facial masks, bath oils, and body washes. Other products, like pharmaceutical compositions (e.g., including an oily active dispersed in an aqueous carrier, e.g., an ointment or a liniment, so-called “fat emulsions” or “lipid emulsions” used, e.g., for intravenous nutritional supplementation, and the like), can also be provided using the emulsions and/or foams of the disclosure. Notably, the starches can provide good emulsification and foaming ability, even while being plant-sourced and biodegradable.

The person of ordinary skill in the art can adapt conventional emulsification and foaming techniques for use in making the emulsions and foams described herein. For example, another aspect of the disclosure is a method for making an emulsion or product as described herein. One such method includes mixing the hydrophobic phase, the hydrophilic phase and an inhibited starch as described herein under conditions sufficient to form the emulsion. Another such method includes mixing the hydrophobic phase, the hydrophilic phase and an unfragmented inhibited starch having a sedimentation volume in the range of 5-50 mL/g and a protein content in the range of 0.2-8 wt. % under conditions sufficient to fragment the starch to a median primary particle size in the range of 0.2-5 microns and to form the emulsion (e.g., under shear). One method for making a foam includes mixing the liquid phase and an inhibited starch as described herein under conditions sufficient to form the foam (e.g., under shear and/or with addition of gas). Another method for making a foam includes mixing the liquid and an unfragmented inhibited starch having a sedimentation volume in the range of 5-50 mL/g and a protein content in the range of 0.2-8 wt. % under conditions sufficient to fragment the starch to a median primary particle size in the range of 0.2-5 microns and to form the foam (e.g., under shear and/or with addition of gas).

These methods will often involve mixing under shear, e.g., in a colloid mill, microfluidizer, homogenizer (e.g., Gaulin, APV), and the like. In various embodiments, the mixing (e.g., under shear) is performed at a temperature of at least 80° C. In various desirable embodiments, the inhibited starch is provided in ungelatinized form, and is gelatinized in the processing of the product, e.g., under the conditions of the mixing, or at a later stage in the processing. In other embodiments, the mixing is performed under conditions that do not gelatinize the starch, but the starch is gelatinized in a later processing step. And in other embodiments, the starch is provided in gelatinized form. The person of ordinary skill in the art can use conventional pregelatinization processes to provide an already-gelatinized starch to a process.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

In this disclosure, “comprises,” “comprising,” “containing,” and “having” and the like can have the meaning ascribed to them in U.S. Patent law and can mean “includes,” “including,” and the like. “Consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. By contrast, the transitional phrase “consisting of” excludes any element, step, or ingredient not specified.

As used in the description herein and throughout the claims that follow, the meaning of “a,” “an,” and “the” includes plural reference unless the context clearly dictates otherwise.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1—Preparation and Characterization of Inhibited Amaranth Starch

Amaranth starch has a small granular size, on the order of about 1 micron. The present inventors determined that an inhibited amaranth starch is useful as an emulsion stabilizer. To that end, amaranth starch was extracted from amaranth flour by an alkaline extraction process. A 0.15 weight percent (wt %) sodium hydroxide solution was prepared by dissolving a weighed amount of sodium hydroxide into a weighed amount of reverse osmosis (RO) water to yield a 0.15 wt % sodium hydroxide solution. Twenty wt % of amaranth flour was added into the 0.15% sodium hydroxide solution with stirring (e.g., an overhead stirrer at about 100 rpm for about 1 hour at ambient temperature). The slurry was then filtered through a U.S. No. 100 sieve screen and the filtered liquid (milk-1) was collected. The fraction on top of the sieve was collected in a beaker, and 0.15% sodium hydroxide solution was added (e.g., the same amount as the amaranth (or other) flour weight) with stirring (e.g., with a spatula) for 10 minutes at ambient temperature. The slurry was filtered through a U.S. No. 100 sieve screen again, and the fraction on top of the sieve was discarded. The filtered liquid (milk-2) was collected.

Milk-1 and milk-2 were combined and filtered through a U.S. No. 230 sieve screen and the filtered liquid (milk-3) was collected.

Milk-3 was centrifuged at 3,000 g for 20 minutes. The supernatant was discarded, and the top brownish layer of protein was removed with a laboratory spatula. The remaining white starch layer was re-suspended in RO water (twice the amount as the flour weight), and the pH was adjusted to 6.0±0.1 with a 1N HCl solution. The mixture was centrifuged at 3,000 g for 20 minutes, after which the top brownish layer of protein was removed with a laboratory spatula. The remaining starch was removed and dried in a forced-air oven at 40° C. for 24 hours.

The extracted amaranth starch sample had protein content (based on starch dry basis) from 0.6% to 3%.

Light scattering can be used to characterize median primary particle size. In this case, the starch suspension sample was analyzed on the Beckman Coulter LS 13320 laser diffraction Particle Size Analyzer equipped with Universal Liquid Module and using standard operating procedure. Two drops of the suspension were added to the instrument and read. Each sample was analyzed in duplicate at room temperature and the results were averaged. SEM (scanning electron microscopy) can also be used to characterize properties of amaranth starch.

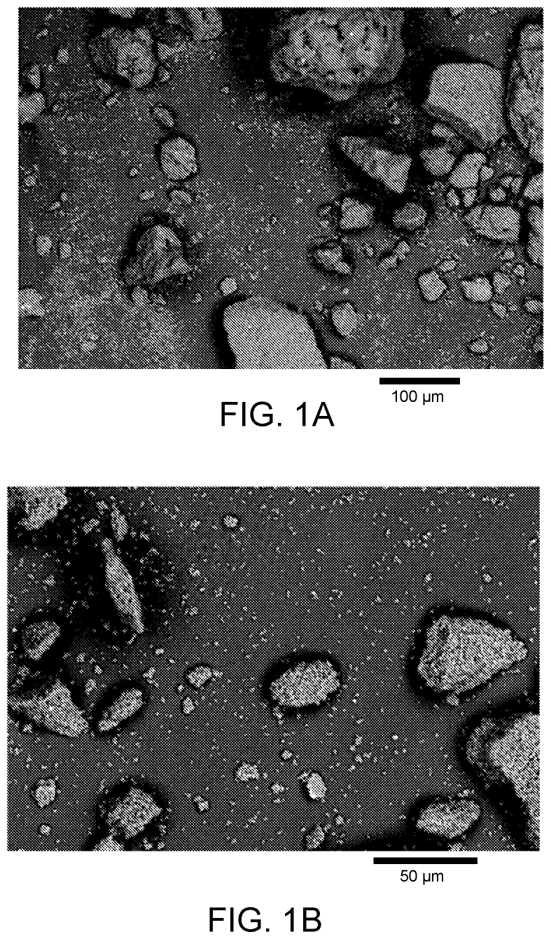

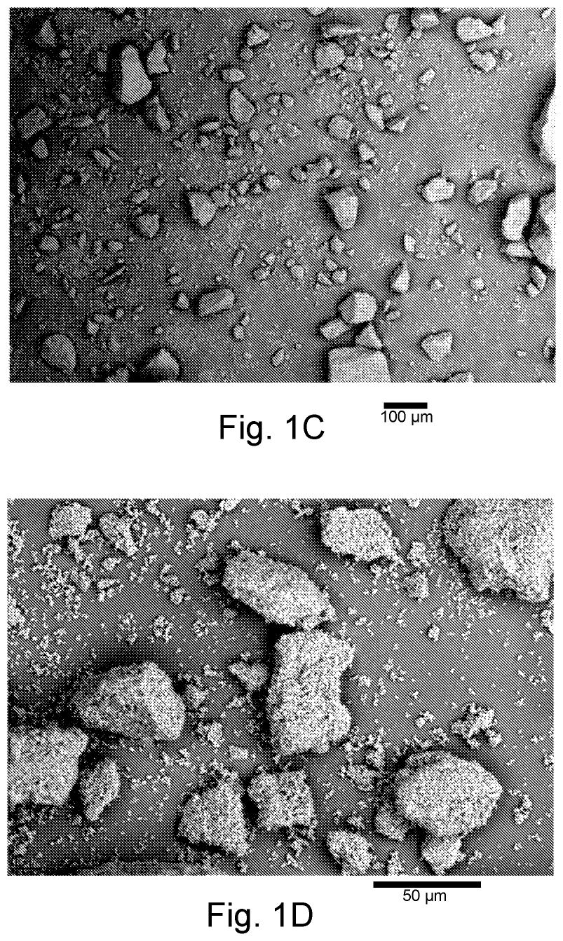

Amaranth starch samples (S/L-5 and S/L-1) were composed of small primary particles (approximately 1 μm) and their aggregates (FIGS. 1A-1D). FIGS. 1A and 1B are S/L-5 at two different magnifications, and FIGS. 1C and 1D are S/L-1 at two different magnifications.

The starches of the disclosure are gelatinized when used as emulsion stabilizers; however, they can be gelatinized before, during, or after emulsification. However, in order to retain starch particle structure, e.g., during the emulsion and gelatinization processes, the starches of the disclosure are inhibited.

Amaranth starches described above were adjusted to acidic pH and heat treated in order to inhibit them to certain sedimentation volumes. The sedimentation volume (as an assessment of relative degree of inhibition), protein content, and RVA viscosity for the native and inhibited starch were measured. Results are described in Table 1, below.

TABLE 1
Characterization of inhibited amaranth starches

| | | | | | RVA |
|---|---|---|---|---|---|
| | | Moisture | Protein | Sed. | End |
| Sample | | Content | Content | Volume | Viscos |
| Lot | Description | (%) | (%) | (mL/g) | (cP) |
| S/L-1 | Native | 5.21 | 0.80 | 100 | 420 |
| | amaranth | | | | |
| | starch | | | | |
| S/L-2 | Inhibited | 6.39 | 0.72 | 22* | 192 |
| | amaranth | | | | |
| | starch | | | | |
| S/L-3 | Inhibited | 6.39 | 0.89 | 23.5* | 335 |
| | amaranth | | | | |
| | starch | | | | |

*After 48 h settling

Protein content of the samples was analyzed using a high temperature combustion method on an Elementar analyzer. The nitrogen in a sample is determined by catalytic combustion (Dumas method) at 900° ° C. in a stream of helium and oxygen gases using Elementar Vario Max CN automated analyzer. The by-products of combustion are chemically scrubbed to remove undesirable gases such as carbon dioxide, carbon monoxide, sulfur dioxide, water vapor, and excess oxygen. The remaining gases, nitrogen oxides, are carried by helium into a reduction chamber (at 830° C.) containing tungsten metal, where they are converted to molecular nitrogen. The nitrogen gas is measured using a thermal conductivity detector (TCD). The TCD signal is scaled against a nitrogen standard (Bovine Serum Albumin) to yield the nitrogen content of the sample. If the sample contains primarily protein, an estimate of the protein content can be obtained by multiplying the nitrogen concentration by an appropriate factor (6.25 in this case). Samples were run two times.

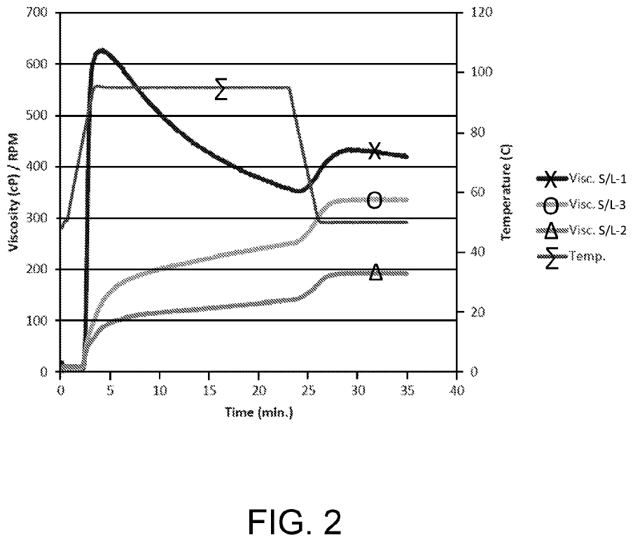

Samples for RVA analysis were prepared as described elsewhere herein, an resulting RVA curves of native and inhibited amaranth starches are shown in FIG. 2. The native amaranth starch (S/L-1) demonstrated a RVA profile of non-inhibited starch, e.g., a peak viscosity while cooked, viscosity instability at hot temperature, and a set-back, or break down, while cooling down. The inhibited amaranth starches (S/L-2 and S/L-3) did not demonstrate a peak viscosity while cooked, but rather consistent viscosity at hot temperature, indicating they have been inhibited.

### Example 2—Emulsification Ability and Emulsion Stability of Uncooked Native, Uncooked Inhibited, and Cooked Inhibited Amaranth Starches

The native and inhibited amaranth starches described above were assessed for their ability to form and maintain emulsions.

Aliquots of S/L-2 and S/L-3 were dispersed at 5% solids in salted pH 6.5 buffer solution (pH 6.5 RVA buffer solution with extra 1% sodium chloride added) and cooked in a 95° C. water bath for 6 minutes under continuous stirring, followed by 20 minutes without stirring. The resulting materials are “cooked inhibited” S/L-2 and S/L-3 as seen below.

To make emulsions, the uncooked starch or cooked starch paste was added to pH 6.5 salted buffer solution. The starch and buffer are then mixed at 5,000 rpm for 1 min using a S25N-25F dispersing tool (with a IKA T25 digital dispenser). The sample is re-mixed at 5,000 rpm for 15 sec, followed by adding oil and mix at 5,000 rpm for 15 sec, thereafter increasing to 11,000 rpm, homogenizing for 90 sec. The mixed starch liquid is then poured into a 100 ml graduated cylinder and left, undisturbed, for stability studies.

The oil phase to aqueous phase ratio was 30/70 (v/v). The (DS) starch content varied from 1, 2, to 4% (w/v) of oil phase. Emulsion recipes are shown in Table 2.

TABLE 2
Formulae for oil-in-water emulsions

| | | | | 5% |
|---|---|---|---|---|
| | | salted | starch | starch |
| | oil | buffer | powder | paste |
| Description (Sample/Lot) | (ml) | (g) | (g) | (g) |
| 1% uncooked inhibited starch (S/L-2) | 30 | 70 | 0.3 | — |
| 1% cooked inhibited starch (S/L-2) | 30 | 64.3 | — | 6 |
| 2% uncooked inhibited starch (S/L-2) | 30 | 70 | 0.6 | — |
| 2% cooked inhibited starch (S/L-2) | 30 | 58.6 | — | 12 |
| 4% uncooked inhibited starch (S/L-2) | 30 | 70 | 1.2 | — |
| 4% cooked inhibited starch (S/L-2) | 30 | 47.2 | — | 24 |
| 1% uncooked native starch (S/L-1) | 30 | 70 | 0.3 | — |
| 2% uncooked native starch (S/L-1) | 30 | 70 | 0.6 | — |
| 4% uncooked native starch (S/L-1) | 30 | 70 | 1.2 | — |
| 1% cooked inhibited starch (S/L-3) | 30 | 64.3 | — | 6 |
| 2% cooked inhibited starch (S/L-3) | 30 | 58.6 | — | 12 |
| 4% cooked inhibited starch (S/L-3) | 30 | 47.2 | — | 24 |

Emulsions were assessed through examination of creamed emulsion phase volume and droplet size via optical microscopy.

Emulsion index (EI) can be used to quantify the emulsification ability and stability using the following equation:

Emulsion index=Hs/He

where Hs is the height of the creamed emulsion and He is the total height of the mixture. The emulsion stability was recorded in Table 3 and Table 4, in which the emulsion index was calculated.

TABLE 3
Emulsification ability and stability quantified by emulsion index

| | Creamed | Total | |
|---|---|---|---|
| | emulsion | mixture | |
| | volume | volume | Emulsion |
| Sample/Lot | (ml) | (ml) | index |
| 1% uncooked inhibited starch (S/L-2) | 0 | 92 | 0 |
| 1% cooked inhibited starch (S/L-2) | 41 | 100 | 0.41 |
| 2% uncooked inhibited starch (S/L-2) | 0 | 94 | 0 |
| 2% cooked inhibited starch (S/L-2) | 45 | 100 | 0.45 |
| 4% uncooked inhibited starch (S/L-2) | 42 | 100 | 0.42 |
| 1% uncooked native starch (S/L-1) | 0 | 100 | 0 |
| 2% uncooked native starch (S/L-1) | 41 | 100 | 0.41 |
| 4% uncooked native starch (S/L-1) | 50 | 100 | 0.50 |

TABLE 4
Emulsification ability and stability quantified
by emulsion index over time

| | Emulsion index | | | | |
|---|---|---|---|---|---|
| | | Day- | Days- | Day- | Day- |
| Sample/Lot | 1 hr | 1 | 3 | 6 | 10 |
| 4% cooked native starch (S/L-1) | 0.6 | 0.49 | 0.48 | 0.465 | 0.45 |
| 1% cooked inhibited starch (S/L-3) | 0.51 | 0.43 | 0.43 | 0.43 | 0.43 |
| 2% cooked inhibited starch (S/L-3) | 0.58 | 0.47 | 0.46 | 0.46 | 0.45 |
| 4% cooked inhibited starch (S/L-3) | 0.67 | 0.57 | 0.57 | 0.55 | 0.54 |

When 4% starch was used, the emulsion prepared by native starch (S/L-1) also had lower emulsion index than emulsions prepared by inhibited starches (S/L-2, S/L-3). Thus, even though the cooked native amaranth starch can stabilize an emulsion (likely due to its protein content), the cooked inhibited amaranth starch can stabilize an emulsion more effectively. Not wishing to be bound by theory, the improved stabilization is thought to derive from the starch particles.

Uncooked native amaranth starch could form emulsion and kept it stable for a week when 2% or 4% starch was used. At one hour after homogenization, oil phase was found in the system containing 1% starch, while not found in the systems containing 2% and 4% starch. The creaming phase was stable after that. The heights of creamed emulsion phase were summarized in Table 4.

Uncooked inhibited amaranth starch was not able to form a stable emulsion. Under each condition, a clear oil phase was observed with only small amount of white foam or emulsion remaining on the top of cylinder less than 2 hours after preparation.

Somewhat surprisingly, cooked inhibited amaranth starch was able to form an emulsion and keep it stable for a week, even when only 1% starch was used. After one hour, no oil phase was found in either system. The creamed emulsion phase was stable after that. The heights of emulsion phase are summarized in Table 3. The emulsification ability and stability of cooked inhibited amaranth starch were higher than that of uncooked native starch, as based on emulsion index.

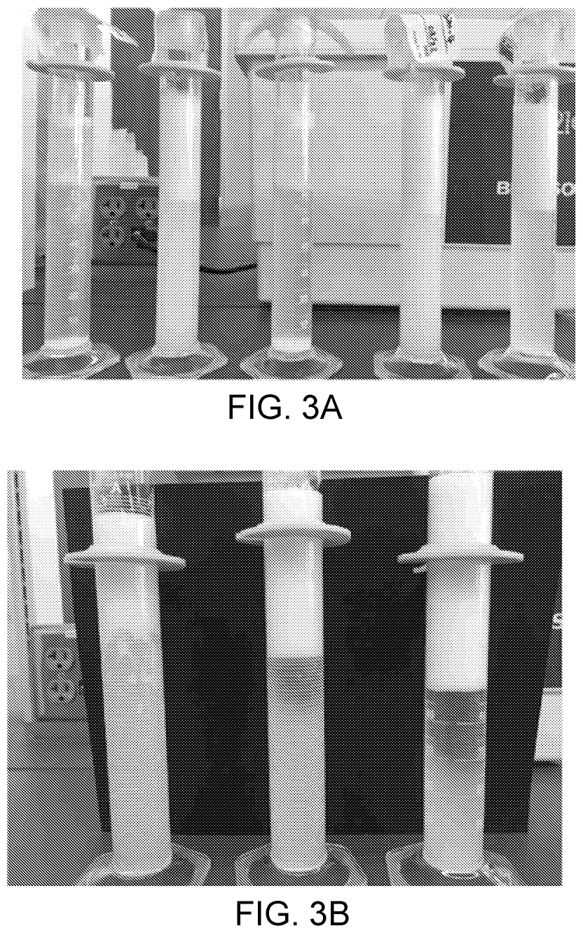

Pictures of creamed emulsions after one week are shown in FIGS. 3A and 3B. FIG. 3A, from left to right, shows 1% uncooked inhibited starch (S/L-2), 1% cooked inhibited starch (S/L-2), 2% uncooked inhibited starch (S/L-2), 2% cooked inhibited starch (S/L-2), and 4% uncooked inhibited starch (S/L-2). FIG. 3B, from left to right, shows 1% uncooked native starch (S/L-1), 2% uncooked native starch (S/L-1), and 4% uncooked native starch (S/L-1).

After one week, emulsion samples were taken from the middle of the creamed emulsion phase by a pipette. One drop of emulsion was put on a glass slide, and a drop of iodine solution was put adjacent to it. As a consequence of mass action, emulsion droplets diffused from the concentrated creamed emulsion to the iodine solution, being simultaneously diluted and stained for optical observation.

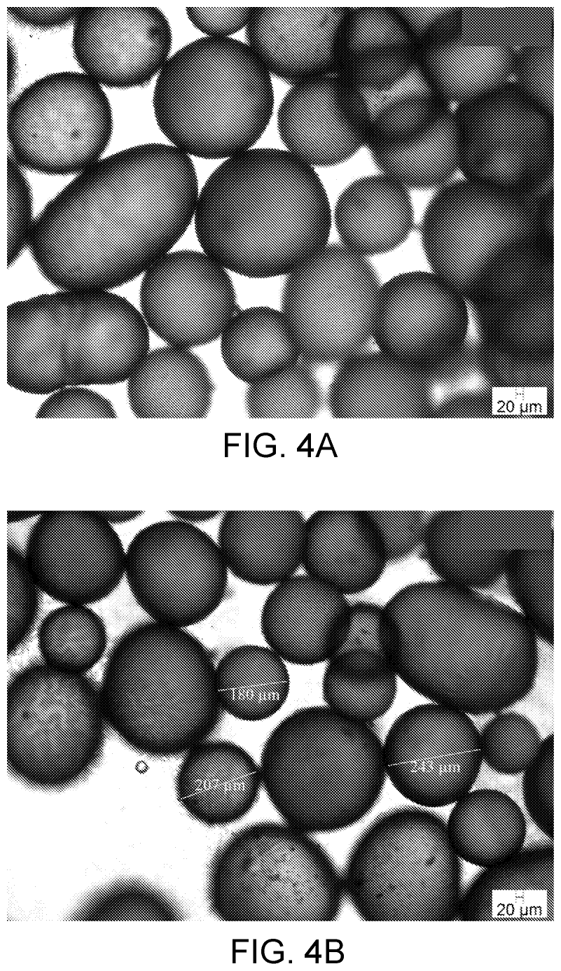

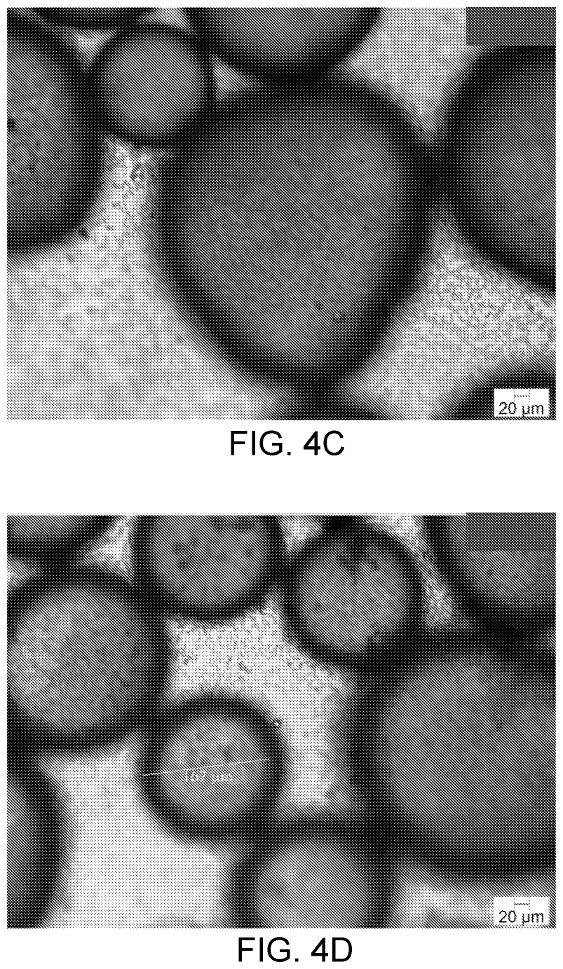

Emulsion droplets stabilized by uncooked native amaranth starch at 2% starch content are shown in FIGS. 4A-4D. The emulsion droplet size was about 200 μm. There were (starch) particles found on the droplet surface (oil-water interface) and stained by iodine to be brownish. The irregular shape of the emulsion droplets was another evidence of Pickering emulsion. Particles in the continuous phase were found as well, indicating the starch particles were not fully attached onto the oil/water interface.

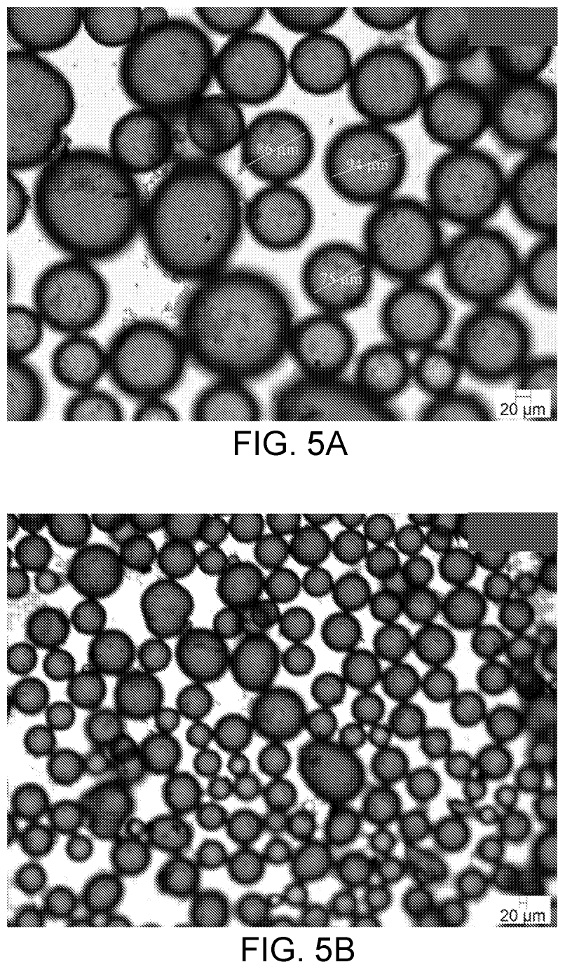

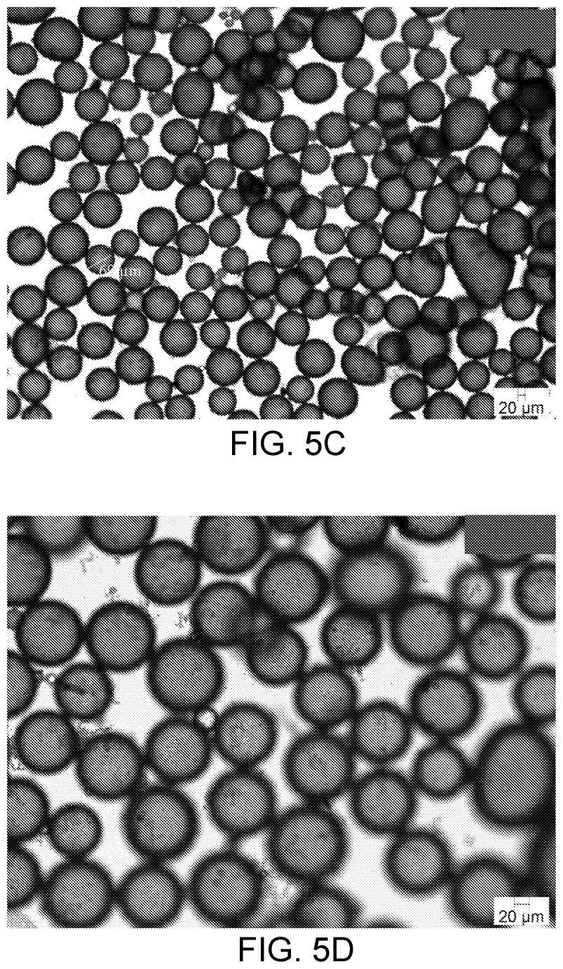

Emulsion droplets stabilized by uncooked native amaranth starch at 4% starch content are shown in FIGS. 5A-5D. The emulsion droplet size was about 90 μm. There were (starch) particles found on the droplet surface and stained by iodine to be brownish. The emulsion droplets with irregular shape was observed as well.

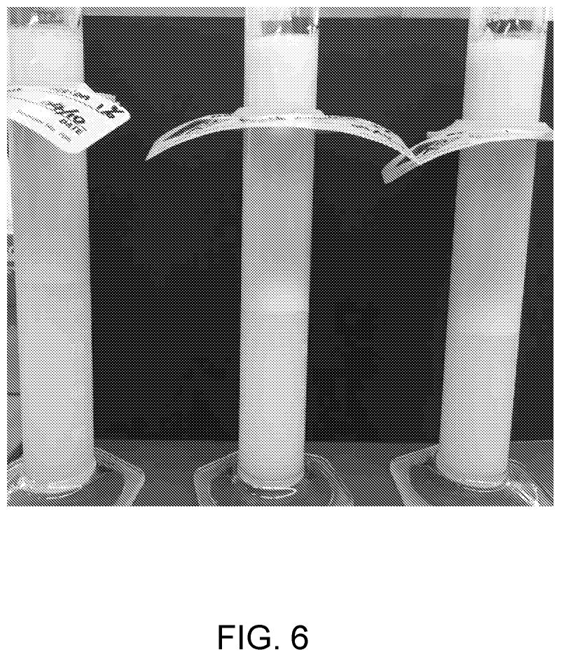

Emulsions stabilized by another sample of cooked inhibited amaranth starch (S/L-2) at 1%, 2% and 4% starch content, after 1.5 hours rest time, are shown in FIG. 6.

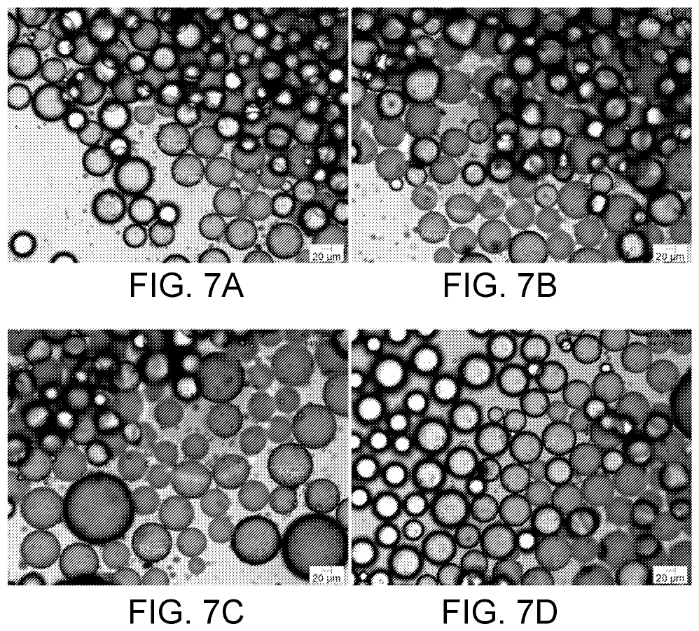

Emulsion droplets stabilized at 1% starch content are shown in FIGS. 7A-7D. The emulsion droplet size was again about 70 μm, though some droplets of a much larger size were again observed. There were (starch) particles found on the droplet surface and stained by iodine to be brownish. Although emulsion droplets with an irregular shape was not observed, the monodispersed emulsion droplet size suggested a Pickering emulsion.

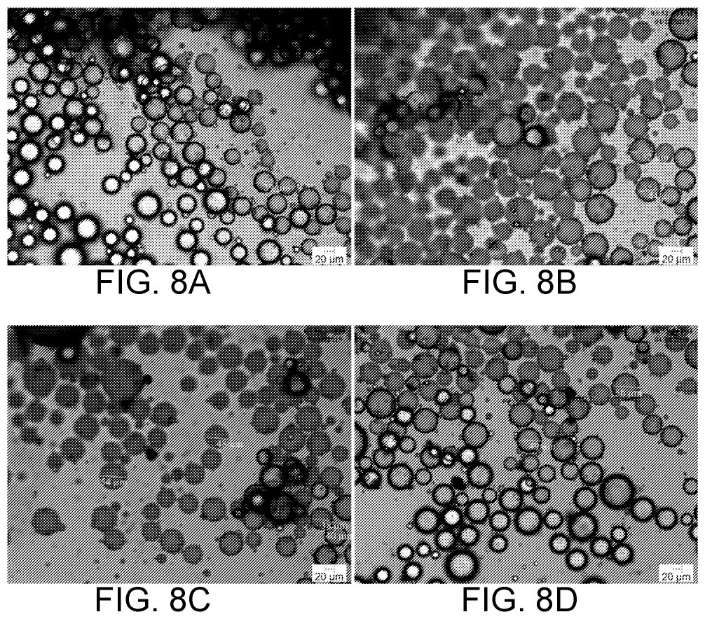

Emulsion droplets stabilized by another sample of cooked inhibited amaranth starch (S/L-2) at 2% starch content were shown in FIGS. 8A-8D. The emulsion droplet size was again about 50 μm, and droplet size distribution tended to be narrow. There were starch particles found on the droplet surface and stained by iodine to be brownish.

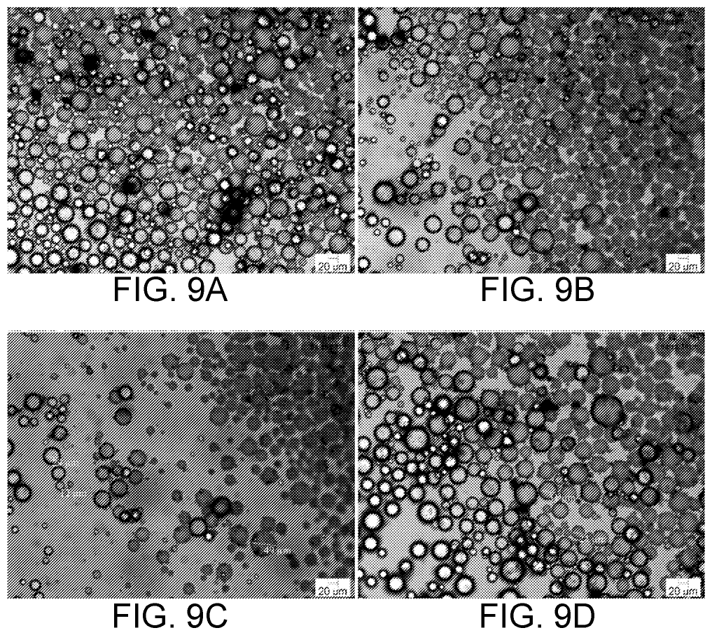

Emulsion droplets stabilized by cooked inhibited amaranth starch (S/L-2) at 4% starch content were shown in FIGS. 9A-9D. The emulsion droplet size was about 25 μm, and droplet size distribution tended to be monodispersed. There were starch particles found on the droplet surface and stained by iodine to be brownish.

Particles were found on the oil-water interface in the emulsions stabilized by both uncooked native amaranth starch and cooked inhibited amaranth starch, indicating the emulsions are particle stabilized. In these experiments, emulsion droplet size decreased when starch content increased. Emulsion droplet size decreased from emulsion stabilized by uncooked native amaranth starch to emulsion stabilized by cooked inhibited amaranth starch.

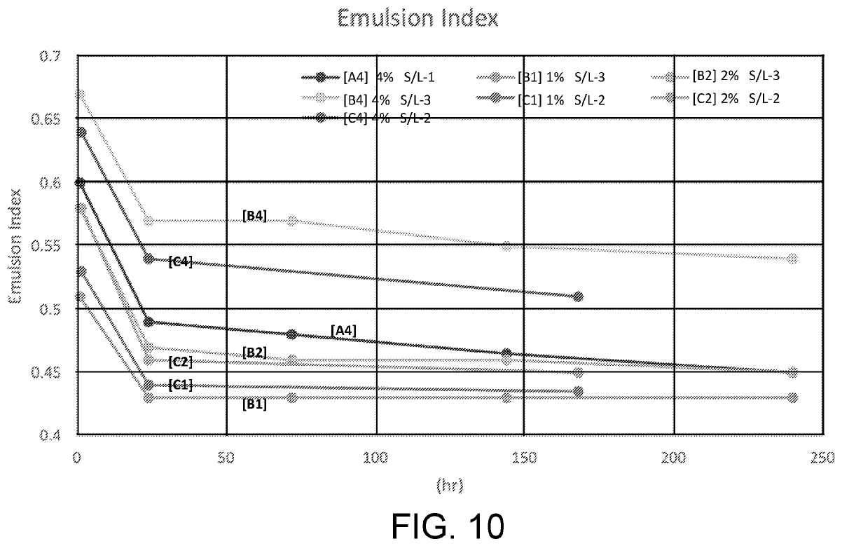

Emulsion index data are plotted in FIG. 10. Both cooked native and cooked inhibited amaranth starches were able to stabilize emulsions. In addition, emulsion droplet size decreased as starch content increased.

### Example 3—Foaming Property of Cooked Inhibited Amaranth Starch

As the emulsification properties of cooked inhibited amaranth starch have been confirmed, the ability of the starch to stabilize the air/water interface (i.e., produce and support foams) was assessed. The foam forming and stabilizing abilities of cooked inhibited amaranth starch (S/L-2 with SV=22 mL/g, RVA end viscosity=192 cP) were assessed.

The starch paste was prepared by cooking the starch at 5% DS in salted pH 6.5 buffer solution in 95° C. water bath for 6 minutes under continuous stirring and 20 minutes steadily. The starch paste was diluted to 2% (w/w) with salted pH 6.5 buffer solution, followed by whipping 100 ml starch paste (2%) in a 500 ml plastic beaker with a Brentwood Hand Mixer (HM-48R) at speed 5 for 10 minutes. The whipped system was poured into a 500 ml graduated cylinder immediately after whipping, and the total volume of the whipped system and the volume of drained serum phase at 5 minutes after whipping were recorded.

The total volume of the whipped system and the volume of drained serum phase were recorded at designated intervals after whipping (e.g., 0.25 hours, 0.5 hours, 1 hour, etc.).

The foam capacity (FC) was determined by measuring the foam volume after whipping as follows:

FC=(V2−V1)/V1×100%

Where V1=volume of solution before whipping, and V2=volume of total content after whipping (at 5 min).

Foam maximum density is determined by measuring the foam volume and liquid volume:

Foam maximum density=(V_{liq(i)}−V_{liq(f)}/V_{foam(f)}

Where V_{liq(i)} and V_{liq(f)} are the initial liquid volume and the liquid volume after whipping (at 5 minutes), V_{foam(f)} is the foam volume after whipping (at 5 minutes).

The foam stability is calculated by using the following equation:

FS=Vₜ/V₀×100

where Vₜ=foam volume at time t, and V₀=initial volume of the dispersion (at 5 minutes).

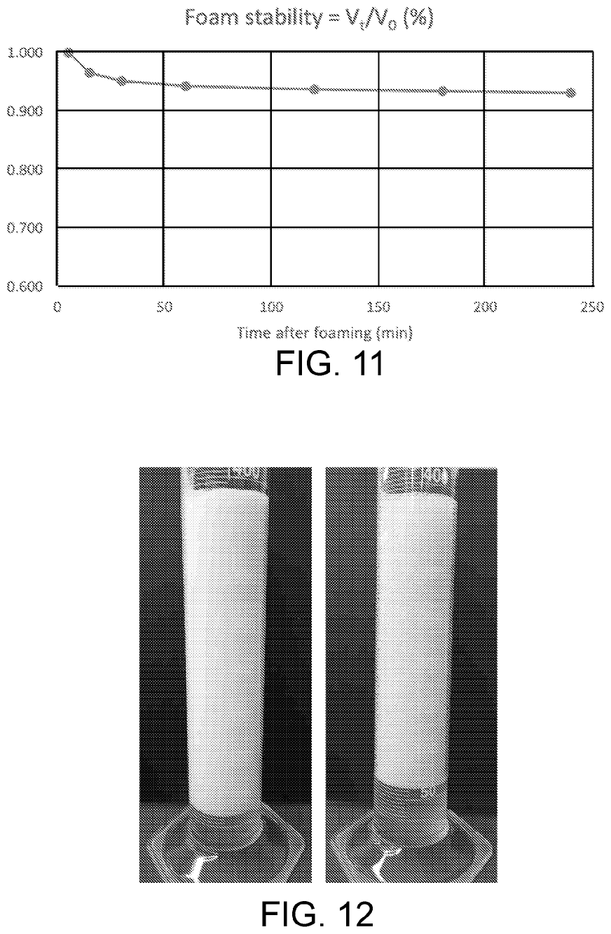

Foam capacity and stability results are summarized in Table 5 and plotted in FIG. 11.

TABLE 5
Foam capacity and foam stability of cooked
inhibited amaranth starch (S/L-2)

| | | | | Foam | |
|---|---|---|---|---|---|
| Time | Serum | Total | Foam | density | Foam |
| (min) | (ml) | (ml) | (ml) | (g/ml) | stability |
| 5 | 40 | 390 | 350 | 0.171 | 1.000 |
| 15 | 52 | 390 | 338 | 0.142 | 0.966 |
| 30 | 57 | 390 | 333 | 0.129 | 0.951 |
| 60 | 60 | 390 | 330 | 0.121 | 0.943 |
| 120 | 62 | 390 | 328 | 0.116 | 0.937 |
| 180 | 63 | 390 | 327 | 0.113 | 0.934 |
| 240 | 64 | 390 | 326 | 0.110 | 0.931 |

The foam capacity of cooked inhibited amaranth starch was calculated to be 290%. The foam maximum density was calculated to be 0.171 g/mL. Foam stability was 93% at 4 hours.

FIG. 12 shows a foam time lapse. The freshly prepared foam (5 minutes after foam preparation at left) had small and tense air cells; therefore, the foam was smooth and white. After 4 hours (at right), the foam became coarse and matte, indicating the partial collapse of smaller air cells into larger air cells, on average.

### Example 4—Preparation of Inhibited Quinoa Starch

Like amaranth starch, quinoa starch exhibits a small particle size in its native form. The present inventors have determined that, like amaranth starch, inhibited quinoa starch can be especially useful in stabilizing emulsions and foams.

Quinoa starch was extracted from quinoa flour by an alkaline extraction process. The isolation of quinoa starch from quinoa flour mirrors the amaranth process.

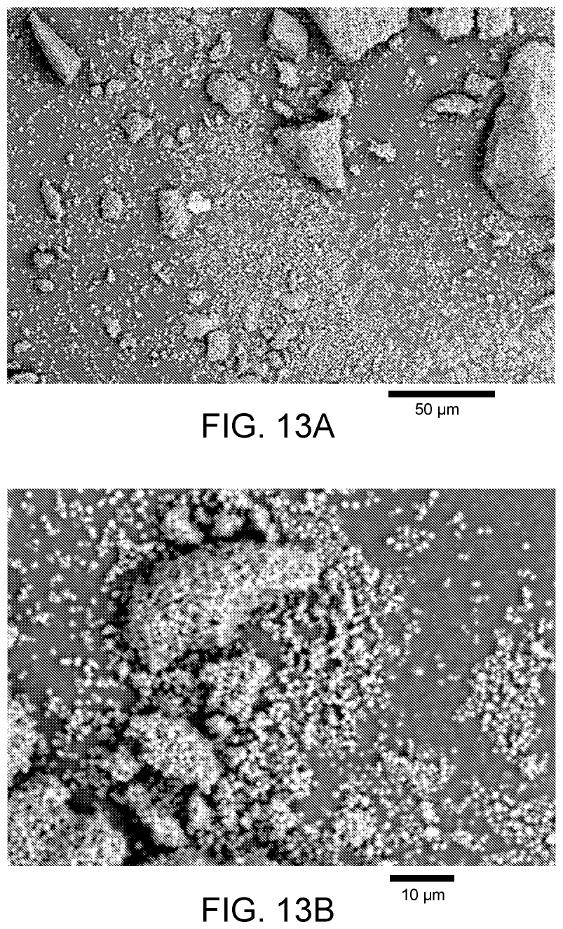

The extracted quinoa starch (S/L-4) had a uniform particle morphology and primary particle size (about 2 microns) shown in SEM images (FIGS. 13A and 13B).

The extracted quinoa starch contained 7.53% moisture, 0.95% protein, 0.79% fat, 0.61% ash and 81.47% starch.

The isolated quinoa starch was inhibited as described above for the amaranth starch.

The sedimentation volume (as an assessment of relative degree of inhibition), and protein content for the native and inhibited starch were measured as described elsewhere herein. Results are described in the Table 6, below.

TABLE 6
Characterization results of heat-treated quinoa starch

| | | | | | RVA |
|---|---|---|---|---|---|
| | | Moisture | Protein | Sedimentation | End |
| Sample/ | | content | content | volume† | Viscos |
| Lot | Description | (%) | (%) | (ml/g) | (cP) |
| S/L-5 | Inhibited | 7.23 | 0.81 | 22 | 153 |
| | quinoa | | | | |
| | starch | | | | |

†after 48 hrs

Samples for SV analysis were prepared as described elsewhere herein, except that sedimentation volume was recorded after 48 hours, rather than 24 hours, in order to provide additional time for the small particles to settle.

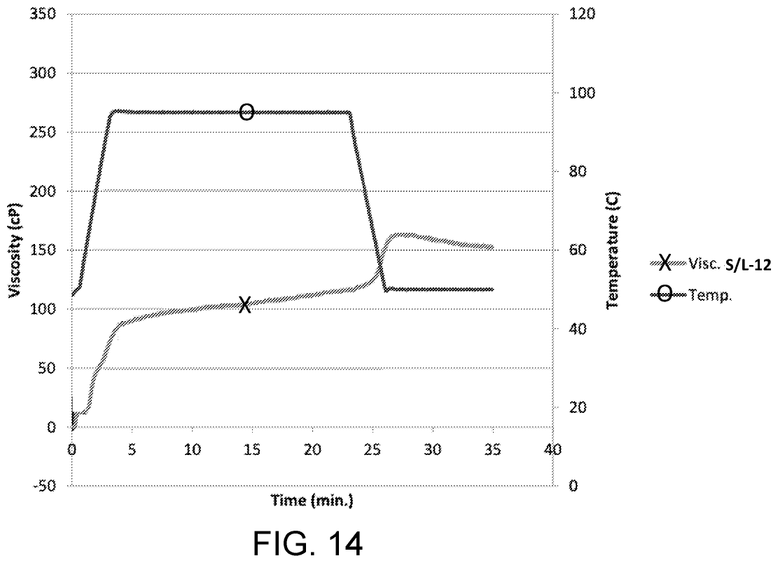

Samples for RVA analysis were prepared as described elsewhere herein, and the resulting RVA curve of heat-treated quinoa starch (S/L-5) is shown in FIG. 14. S/L-5 starch demonstrated an RVA profile similar to an inhibited starch, and RVA final viscosity was measured to be 153 cP.

### Example 5—Emulsion and Foam Stabilized by Inhibited Quinoa Starch

Aliquots of S/L-5 were dispersed at 5% DS in salted pH 6.5 buffer solution (pH 6.5 RVA buffer solution with extra 1% sodium chloride added) and cooked in a 95° C. water bath for 6 minutes under continuous stirring, followed by 20 minutes without stirring. The resulting materials are the “cooked inhibited” S/L-5 seen below.

Cooked inhibited S/L-5 was used for emulsification tests. The (vegetable) oil to water ratio was 30/70 (v/v), and the S/L-5 (DS) starch content varied from 1, 2, to 4% (w/v) of oil phase. Emulsion recipes are shown in Table 7. Methods were as described above for the amaranth starch.

TABLE 7
Formulae for oil-in-water emulsions

| | | | 5% DS |
|---|---|---|---|
| | | salted | starch paste |
| Sample | oil (ml) | buffer (g) | (g) |
| 1% cooked inhibited S/L-5 | 30 | 64.3 | 6 |
| 2% cooked inhibited S/L-5 | 30 | 58.6 | 12 |
| 4% cooked inhibited S/L-5 | 30 | 47.2 | 24 |

Emulsions and foams were assessed through examination of emulsion index and optical microscopy.

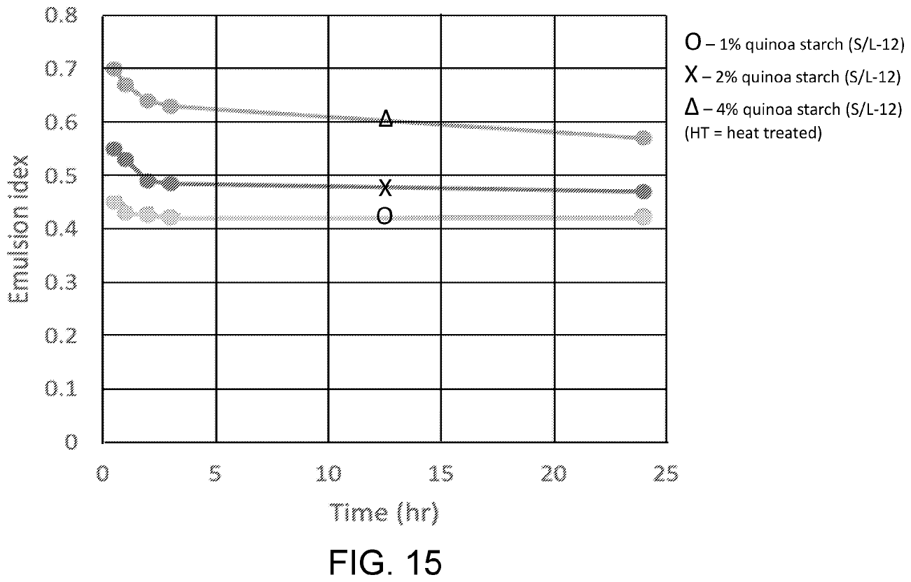

Emulsions according to Table 7 above were prepared as described elsewhere herein, and emulsion index was calculated at 0.25, 0.5, 1, 2, 3 and 24 hours after initial emulsion, and those emulsion indices were plotted against storage time in FIG. 15. Cooked inhibited S/L-5 quinoa starch demonstrated good emulsion ability and stability, each decreasing with decreasing starch concentration. For example, the emulsion ability (i.e. emulsion index at 1 hour) decreased from 0.67 to 0.43 when the starch concentration decreased from 4% to 1%. The emulsion stability also decreased along with the decrease of starch concentration. After two days, emulsion samples were taken in the middle of emulsion phase, by pipette, for an optical microscopy study. Free oil droplets were observed in the systems containing 1% and 2% HT S/L-5, while homogeneous oil-in-water emulsion was observed in the system containing 4% S/L-5, indicating the latter was more stable.

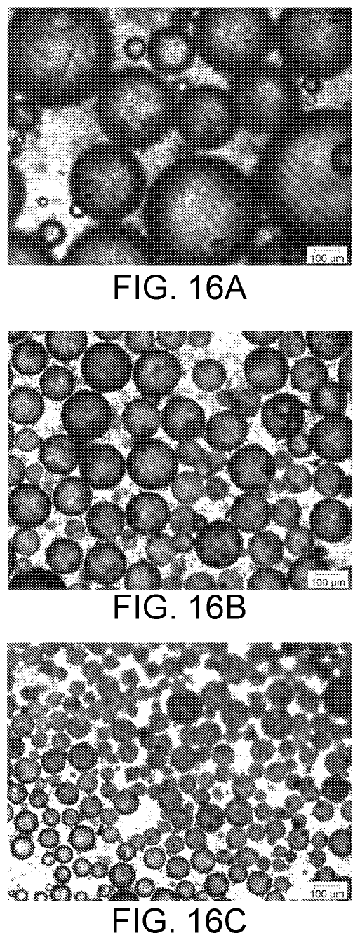

FIGS. 16A-16C are a series of micrographs of emulsion droplets of heat-treated quinoa starch (S/L-5) after two days at 1%, 2% and 4% concentration, respectively. Optical microscopic observation of emulsion droplets stabilized by cooked inhibited S/L-5 two days after preparation showed that the oil-water interface was surrounded by particles in all samples, with the emulsion droplet size decreasing along with increasing S/L-5 starch concentration.

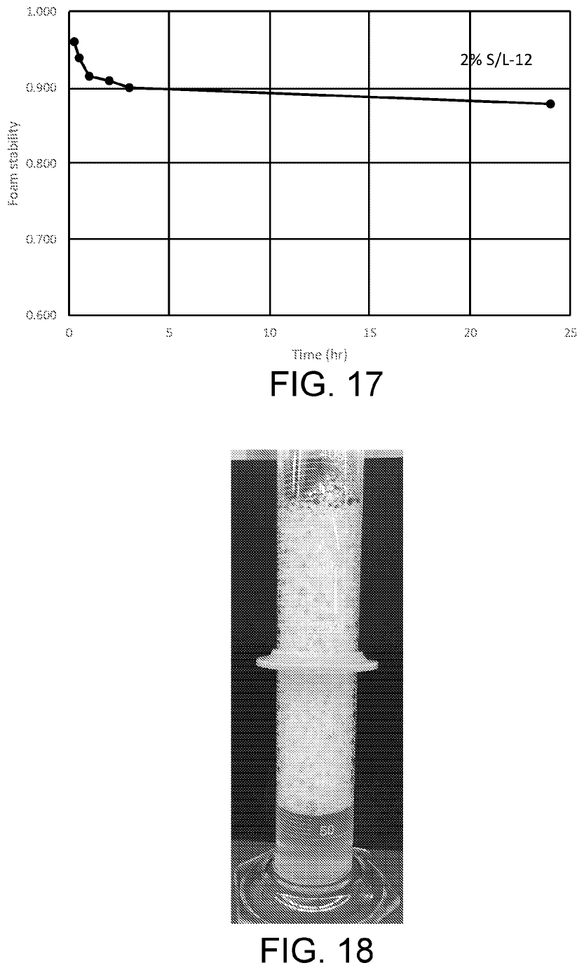

Foams were prepared according to the procedures shown in Example 3. The foaming capacity, foam maximum density, and foam stability are summarized in Table 8. The foam stability of cooked inhibited S/L-5 starch pastes was plotted in FIG. 17. The S/L-5, at 2% (wt), could stabilize foam significantly.

TABLE 8
Foaming performances of citrated and heat-treated quinoa starches.

| | Foam | Foam max | Foam stability | | | | | |
|---|---|---|---|---|---|---|---|---|
| Sample | capacity | density | 0.25 hr | 0.5 hr | 1 hr | 2 hrs | 3 hrs | 24 hrs |
| 2% cooked | 270 | 0.182 | 0.961 | 0.939 | 0.915 | 0.909 | 0.900 | 0.879 |
| inhibited | | | | | | | | |
| S/L-5 | | | | | | | | |

The foams were coarse 24 hours after preparation; however, they were not collapsed (FIG. 18).

### Example 6—Emulsions Stabilized by Fragmented Corn Starch

First, inhibited waxy corn starches of different sedimentation volumes were fragmented and used to stabilize oil-in-water emulsions.

Inhibited waxy corn starches were provided:

| | Sedimentation | Protein content | RVA Viscosity |
|---|---|---|---|
| Sample | volume | (%, dsb) | (cP) |
| 6A | 29.4 | 1.2 | ~830 |
| 6B | 25.8 | 1.3 | ~300 |
| 6C | 23.9 | 1.3 | ~190 |

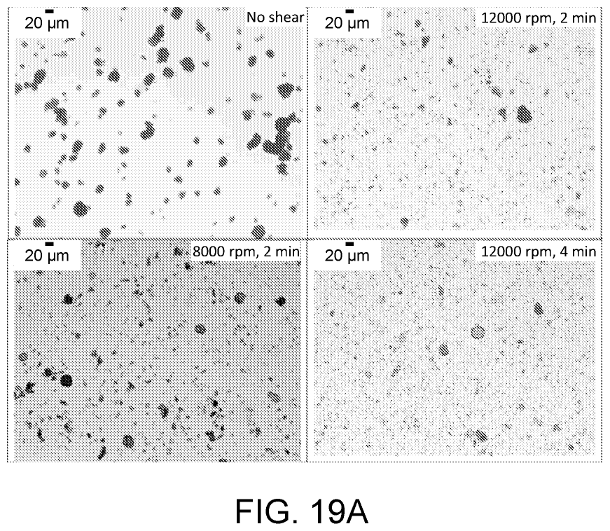

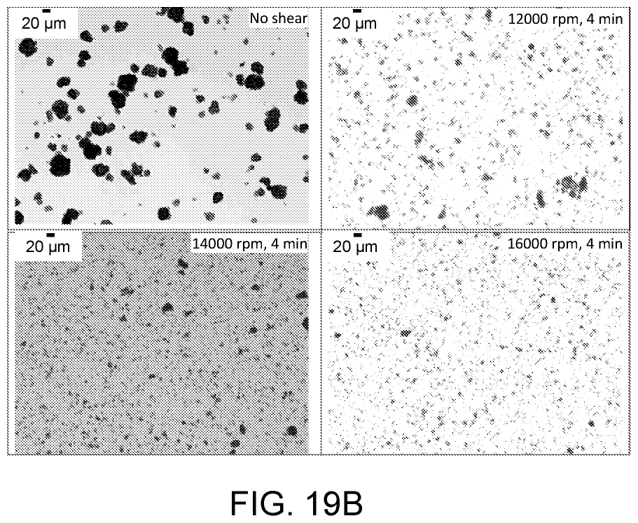

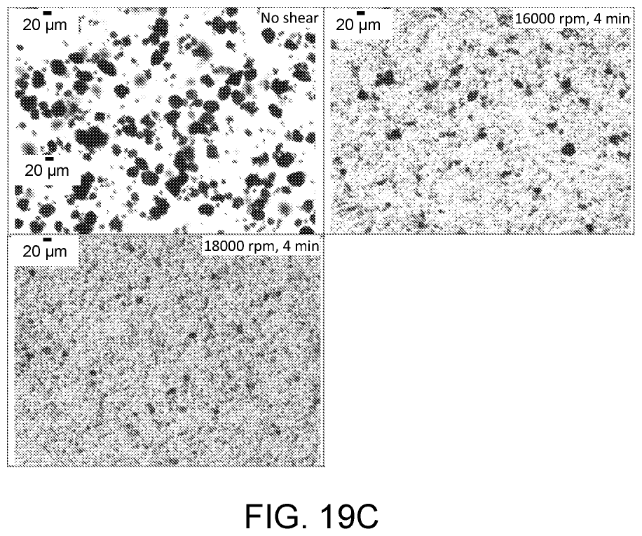

Each of the Samples 6A, 6B, and 6C was cooked at 5% solids in pH 6.5 RVA buffer including 1% salt for 26 minutes (6 minutes with stirring, 20 minutes without) at 95 C. Paste samples were exposed to different degrees of shearing (different times and rpms using an IKA Ultra-Turrax T25 Homogenizer with S25N-25F dispersing tool). Pastes were returned to 5% solids after cooking by addition of any necessary water to account for evaporation. FIGS. 19A, 19B and 19C provide micrographs of starches before and after shearing, respectively, for Sample 6A, Sample 6B and Sample 6C.

Emulsions having variously 6% starch to oil phase, 8% starch to oil phase, and 10% starch to oil phase were prepared at 100 g scale from Samples 6A, 6B and 6C, using amounts shown in the table below:

| | Veg. oil (g) | Water (g) | Starch paste (g) | Na Benzoate (g) |
|---|---|---|---|---|
| 6% | 30 | 33.9 | 36 | 0.1 |
| 8% | 30 | 21.9 | 48 | 0.1 |
| 10% | 30 | 9.9 | 60 | 0.1 |

First water, sodium benzoate, the 5% DS starch paste were combined and adjusted to pH 3.65 using 4N HCl or 5% NaOH. Vegetable oil was added. Mixing using an Ultra-Turrax Homogenizer at 5000 rpm for 2 minutes provided a pre-emulsion, which was then emulsified at 10000 rpm for 3 minutes. Samples were stored at 35° C., and examined using microscopy and a Beckman Coulter LS 13 320 particle size analyzer immediately, after 2 days and after 7 days. A Turbiscan AGS emulsion stability analyzer was used to determine emulsion stability at 35 C after 7 days.

The emulsions were as in the list below:
6A-1 (Sample 6A, unsheared, 6% oil)
6A-2 (Sample 6A, shear 8000 rpm/2 min, 6% oil)
6A-3 (Sample 6A, shear 12000 rpm/2 min, 6% oil)
6A-4 (Sample 6A, shear 12000 rpm/4 min, 6% oil)
6B-1 (Sample 6B, shear 12000 rpm/4 min, 6% oil)
6B-2 (Sample 6B, shear 14000 rpm/4 min, 6% oil)
6B-3 (Sample 6B, shear 16000 rpm/4 min, 6% oil)
6B-4 (Sample 6B, shear 16000 rpm/4 min, 8% oil)
6C-1 (Sample 6C, shear 16000 rpm/4 min, 6% oil)
6C-2 (Sample 6C, shear 18000 rpm/4 min, 8% oil)
6C-3 (Sample 6C, shear 18000 rpm/4 min, 10% oil)

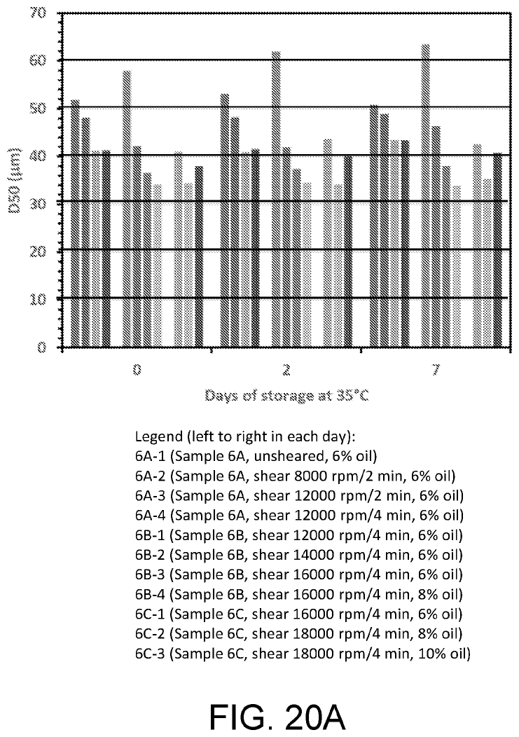

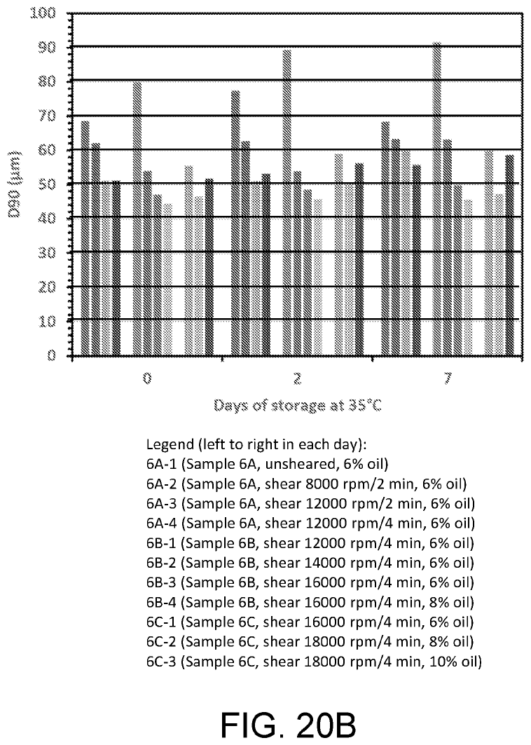

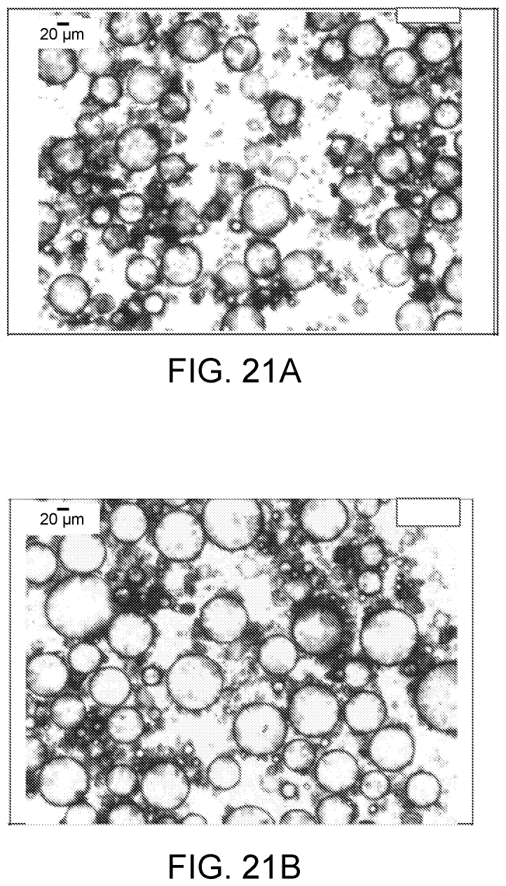

Mean and mode sizes of the emulsion droplets are shown respectively in FIGS. 20A and 20B, measured on the day of emulsification, 2 days after emulsification, and 7 days after emulsification. FIGS. 21A and 21B are micrographs of Emulsion 6B-1, respectively on the day of preparation and after 7 days storage at 35° C.

These data suggest that oil droplet size generally decreases with increased shear. At constant shear, a starch with a lower sedimentation volume (i.e., more highly inhibited) generates larger oil droplets. Higher relative amounts of starch provided smaller droplet size. Even the unsheared starch sample (Emulsion 6A-1) appeared to stabilize the emulsion; the inventors surmise that the starch fragmented during emulsification. This demonstrates that fragmentation during emulsification is also a viable method to make fragmented inhibited starch-stabilized emulsions.

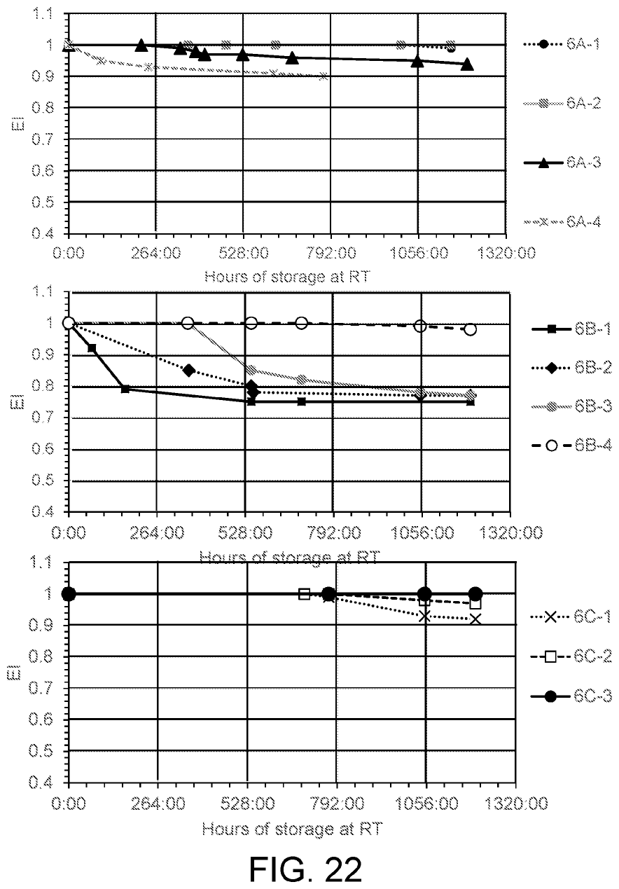

Emulsion index was also measured, as shown in FIG. 22. These samples were stored at laboratory temperature over the course of the experiment. Many of the emulsions were stable (EI>0.9) over 60 days. Without intending to be bound by theory, the present inventors believe that these results are due to the effect of inhibition and degree of fragmentation on water holding and/or viscosity of the starches.

### Example 7—Emulsions Stabilized by Fragmented Corn Starch—Protein Content

In this Example, inhibited waxy corn starches of a different protein content than those of Example 6 were fragmented and used to stabilize oil-in-water emulsions.

Inhibited waxy corn starches were provided:

| | Sedimentation | Protein content | RVA Viscosity |
|---|---|---|---|
| Sample | volume | (%, dsb) | (cP) |
| 7A | 28 | 2.1 | ~600 |
| 7B | 22 | 2.2 | ~250 |

Each of Samples 7A and 7B was cooked at 5% solids as described above in Example 6. in pH 6.5 RVA buffer including 1% salt for 26 minutes (6 minutes with stirring, 20 minutes without) at 95° C. Paste samples were sheared under various conditions generally as described above in Example 6.

Emulsions having variously 6% starch to oil phase and 8% starch to oil phase were prepared as described above in Example 6. The emulsions were as in the list below:
7A-1 (Sample 7A, shear 12000 rpm/4 min, 6% oil)
7A-2 (Sample 7A, shear 20000 rpm/4 min, 8% oil)
7A-3 (Sample 7A, shear 24000 rpm/4 min, 8% oil)
7B-1 (Sample 7B, shear 18000 rpm/4 min, 8% oil)
7B-2 (Sample 7B, shear 20000 rpm/4 min, 8% oil)
7B-3 (Sample 7B, shear 24000 rpm/4 min, 8% oil)

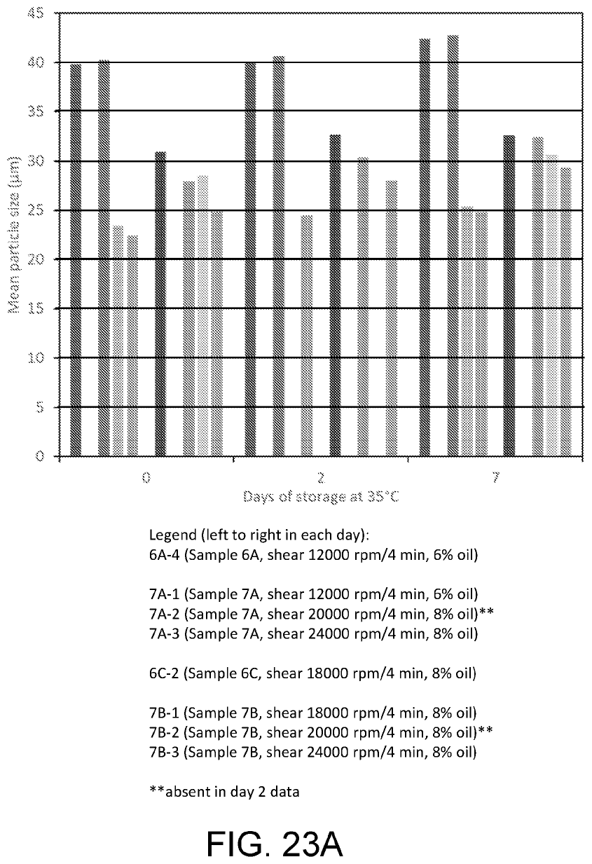

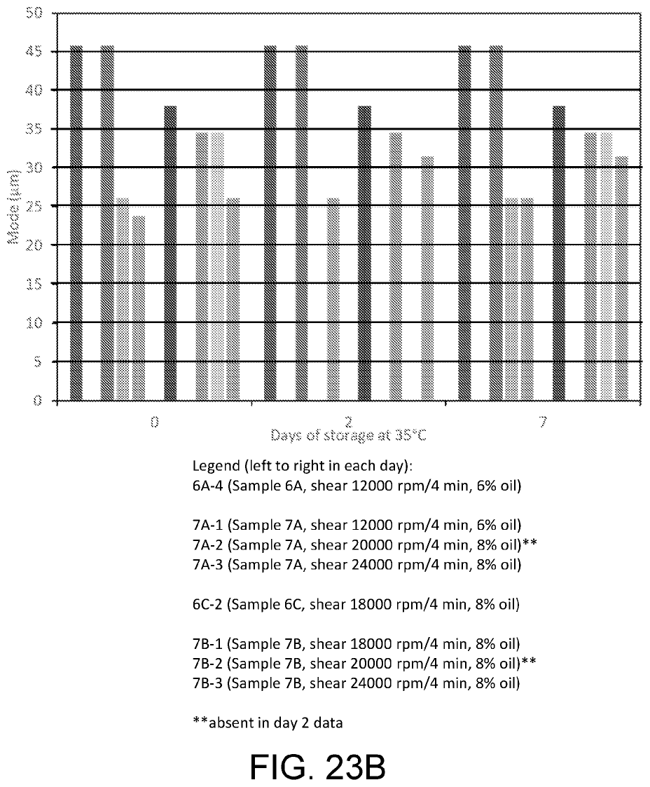

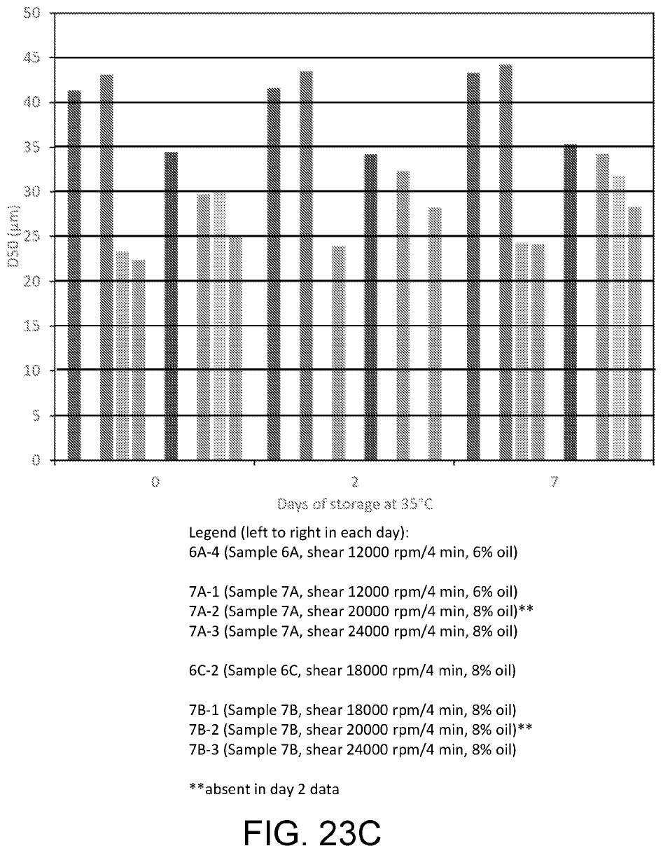

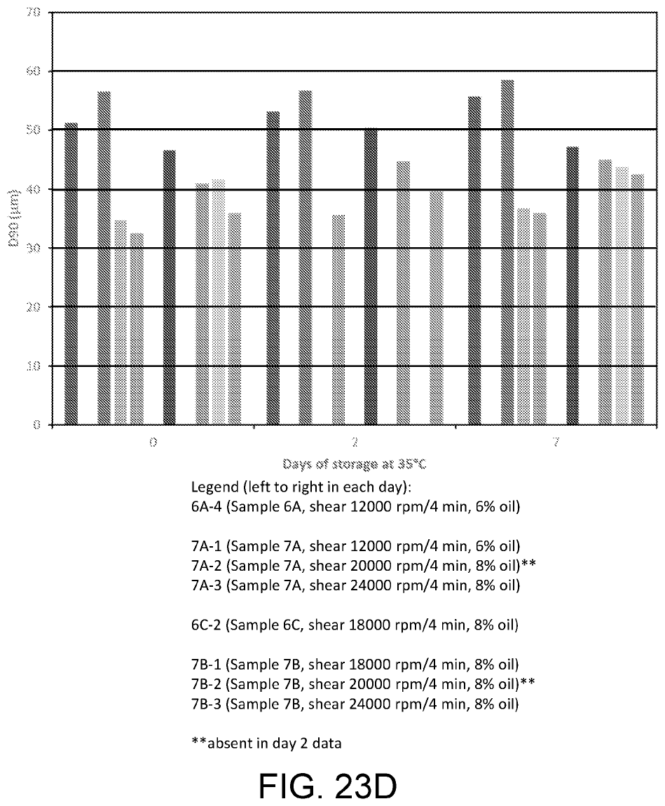

Mean and mode sizes of the emulsion droplets are shown respectively in FIGS. 23A and 23B, measured on the day of emulsification, 2 days after emulsification, and 7 days after emulsification. D50 and D90 sizes of the emulsion droplets are shown respectively in FIGS. 23C and 23D, measured on the day of emulsification, 2 days after emulsification, and 7 days after emulsification. Data are plotted with data for Emulsions 6A-4 and 6C-2 from Example 6, for comparison.

These data suggest that the change from ˜1% protein to ˜2% protein in starches with similar inhibition levels does not have a strong effect on droplet size distribution. But these data confirm the stability of the emulsions, with mean, mode, D50 and D90 droplet sizes staying relatively stable over a week of storage.

Additional aspects and of the disclosure are provided by the enumerated embodiments below, which can be combined in any number and in any combination not technically or logically inconsistent.
Embodiment 1. An inhibited starch having a protein content in the range of 0.2-8 wt % on a dry starch basis.
Embodiment 2. An inhibited starch according to embodiment 1, having a median primary particle size in the range of 0.2-5 microns.
Embodiment 3. An inhibited starch according to embodiment 1 or embodiment 2, wherein the starch is an amaranth starch.
Embodiment 4. An inhibited starch according to embodiment 1 or embodiment 2, wherein the starch is a quinoa starch.
Embodiment 5. An inhibited starch according to embodiment 1 or embodiment 2, wherein the starch is a rice starch.
Embodiment 6. An inhibited starch according to embodiment 1 or embodiment 2, wherein the starch is a fragmented starch, e.g., a fragmented corn starch.
Embodiment 7. An inhibited starch according to any of embodiments 1-6, having a median primary particle size in the range of 0.2-2 microns, e.g., 0.2-1 micron.
Embodiment 8. An inhibited starch according to any of embodiments 1-6, having a median particle size in the range of 0.5-5 microns, e.g., 0.5-2 microns. or 1-5 microns.
Embodiment 9. An inhibited starch according to any of embodiments 1-8, wherein at least 50%, e.g., at least 75%, or even at least 90%, of the primary particles of the inhibited starches have particle sizes in the range of 0.2-5 microns, e.g., 0.2-2 microns, or 0.2-1 micron.
Embodiment 10. An inhibited starch according to any of embodiments 1-8, wherein at least 50%, e.g., at least 75%, or even at least 90%, of the primary particles of the inhibited starches have particle sizes in the range of 0.5-5 microns, e.g., 0.5-2 microns, or 1-5 microns.
Embodiment 11. An inhibited starch according to any of embodiments 1-5 and 7-10, having a sedimentation volume in the range of 5-50 mL/g.
Embodiment 12. An inhibited starch according to any of embodiments 1-5 and 7-10, having a sedimentation volume in the range of 10-50 mL/g, e.g., 15-50 mL/g, or 20-50 mL/g, or 25-50 mL/g.
Embodiment 13. An inhibited starch according to any of embodiments 1-5 and 7-10, having a sedimentation volume in the range of 5-40 mL/g, 10-40 mL/g, or 15-40 mL/g, or 20-40 mL/g, or 25-40 mL/g.
Embodiment 14. An inhibited starch according to any of embodiments 1-5 and 7-10, having a sedimentation volume in the range of 5-35 mL/g, 10-35 mL/g, or 15-35 mL/g, or 20-35 mL/g, or 25-35 mL/g.
Embodiment 15. An inhibited starch according to any of embodiments 1 and 6-10, wherein the starch is a fragmented starch, e.g., a fragmented corn starch, that is inhibited using conditions that provide a sedimentation volume in the range of 5-50 mL/g to a corresponding un-fragmented starch.
Embodiment 16. An inhibited starch according to embodiment 15, wherein the sedimentation volume is in the range of 10-50 mL/g, e.g., 15-50 mL/g, or 20-50 mL/g, or 25-50 mL/g.
Embodiment 17. An inhibited starch according to embodiment 15, wherein the sedimentation volume is in the range of 5-40 mL/g, 10-40 mL/g, or 15-40 mL/g, or 20-40 mL/g, or 25-40 ml/g.
Embodiment 18. An inhibited starch according to embodiment 15, wherein the sedimentation volume is in the range of 5-35 mL/g, 10-35 mL/g, or 15-35 mL/g, or 20-35 mL/g, or 25-35 mL/g.
Embodiment 19. An inhibited starch according to any of embodiments 1-18, having a protein content in the range of 0.2-5 wt % protein, e.g., in the range of 0.2-3 wt % on a dry starch basis.
Embodiment 20. An inhibited starch according to any of embodiments 1-18, having a protein content in the range of 0.3-8 wt % protein, e.g., in the range of 0.3-5 wt %, or 0.3-3 wt % on a dry starch basis.
Embodiment 21. An inhibited starch according to any of embodiments 1-18, having a protein content in the range of 0.4-8 wt % protein, e.g., in the range of 0.4-5 wt %, or 0.4-3 wt % on a dry starch basis.
Embodiment 22. An inhibited starch according to any of embodiments 1-18, having a protein content in the range of 0.5-8 wt % protein, e.g., in the range of 0.5-5 wt %, or 0.5-3 wt % on a dry starch basis.
Embodiment 23. An inhibited starch according to any of embodiments 1-18, having a protein content in the range of 0.6-8 wt % protein, e.g., in the range of 0.6-5 wt %, or 0.6-3 wt %, or 0.7-8 wt %, or 0.7-5 wt %, or 0.7-3 wt % on a dry starch basis.
Embodiment 24. An inhibited starch according to any of embodiments 1-18, having a protein content in the range of 1-8 wt % protein, e.g., in the range of 1-5 wt %, or 1-3 wt % on a dry starch basis.
Embodiment 25. The inhibited starch product of any of embodiments 1-24, wherein the inhibited starch product has a viscosity at 5% solids in the range of 50-1500 cP in an RVA test.
Embodiment 26. The inhibited starch product of any of embodiments 1-24, wherein the inhibited starch product has a viscosity in the range of 50-1000 cP, 50-850 cP, 50-700 cP, 50-500 cP, 50-400 cP, 50-300 cP, 50-200 cP, 100-1100 cP, 100-1000 cP, 100-850 cP, 100-700 cP, 100-500 cP, 100-400 cP, 100-300 cP, 200-1100 cP, 200-1000 cP, 200-850 cP, 200-700 cP, 200-500 cP, 400-1100 cP, 400-1000 cP, 400-850 cP, 400-700 cP, 600-1100 cP, 600-850 cP, 700-1500 cP, or 700-1300 cP in an RVA test at 5% solids.
Embodiment 27. An inhibited starch according to any of embodiments 1-26, which is chemically inhibited, e.g., by crosslinking with phosphate, adipate, acrolein or epichlorohydrin.
Embodiment 28. An inhibited starch according to any of embodiments 1-26, which is not chemically inhibited.
Embodiment 29. An inhibited starch according to embodiments 1-26, which is not inhibited by crosslinking with phosphate, adipate, acrolein or epichlorohydrin.
Embodiment 30. An inhibited starch according to any of embodiments 1-29, which is thermally inhibited.
Embodiment 31. An inhibited starch according to any of embodiments 1-30, which is not acetylated.
Embodiment 32. An inhibited starch according to any of embodiments 1-31, which is not adipated.
Embodiment 33. An inhibited starch according to any of embodiments 1-32, which is not hydroxyethylated.
Embodiment 34. An inhibited starch according to any of embodiments 1-33, which is not hydroxypropylated.
Embodiment 35. An inhibited starch according to any of embodiments 1-34, which is not carboxymethylated.
Embodiment 36. An inhibited starch according to any of embodiments 1-35, which is not phosphated.
Embodiment 37. An inhibited starch according to any of embodiments 1-36, which is not succinated and does not include fatty acid residues.
Embodiment 38. The inhibited starch of any of embodiments 1-37, wherein the inhibited starch product is not cationic or zwitterionic.
Embodiment 39. An inhibited starch according to any of embodiments 1-38, which is not bleached or oxidized, e.g., with peroxide or hypochlorite.
Embodiment 40. An inhibited starch according to any of embodiments 1-30, which is chemically modified, e.g., one or more of succinated (e.g., octenylsuccinated), acetated, adipated, hydroxyethylated, hydroxypropylated, carboxymethylated, or oxidized.
Embodiment 41. An inhibited starch according to any of embodiments 1-40, wherein the inhibited starch has a relatively low color, i.e., a Yellowness Index of no more than 10.
Embodiment 42. An inhibited starch according to any of embodiments 1-40, wherein the inhibited starch has a relatively low color, i.e., a Yellowness Index of 3-10 or 5-10.
Embodiment 43. An inhibited starch according to any of embodiments 1-40, wherein the inhibited starch has an especially low color, i.e., a Yellowness Index of no more than 8.
Embodiment 44. An inhibited starch product according to any of embodiments 1-43, which is not gelatinized.
Embodiment 45. An inhibited starch according to any of embodiments 1-43, which is gelatinized.
Embodiment 46. An emulsion comprising an emulsified phase that is a hydrophobic phase emulsified within a hydrophilic phase, stabilized by a gelatinized inhibited starch according to embodiment 45.
Embodiment 47. An emulsion comprising an emulsified phase that is a hydrophobic phase emulsified within a hydrophilic phase, stabilized by a gelatinized inhibited starch, wherein the gelatinized inhibited starch has a median primary particle size in the range of 0.2-5 microns and a protein content in the range of 0.2-8 wt. %.
Embodiment 48. An emulsion according to embodiment 47, wherein the gelatinized inhibited starch is a rice starch, an amaranth starch, a quinoa starch or a rice starch.
Embodiment 49. An emulsion according to embodiment 47 or embodiment 48, wherein the gelatinized inhibited starch is a gelatinized form of an inhibited unfragmented starch that has a sedimentation volume of 5-50 mL/g.
Embodiment 50. An emulsion according to embodiment 47 or embodiment 48, wherein the gelatinized inhibited starch is a gelatinized form of an inhibited unfragmented starch that has a sedimentation volume as described in any of embodiments 12-14.
Embodiment 51. An emulsion according to embodiment 47, wherein the gelatinized inhibited starch is a fragmented starch, e.g., a fragmented corn starch.
Embodiment 52. An emulsion according to embodiment 51, wherein the gelatinized inhibited starch is a gelatinized form of a fragmented starch that that is inhibited using conditions that provide a sedimentation volume in the range of 5-50 mL/g to a corresponding un-fragmented starch.
Embodiment 53. An emulsion according to embodiment 52, wherein the sedimentation volume is as described in any of embodiments 16-18.
Embodiment 54. An emulsion according to any of embodiments 47-53, wherein the gelatinized inhibited starch has a median primary particle size in the range of 0.2-2 microns, e.g., 0.2-1 micron.
Embodiment 55. An emulsion according to any of embodiments 47-53, wherein the gelatinized inhibited starch has a median particle size in the range of 0.5-5 microns, e.g., 0.5-2 microns. or 1-5 microns.
Embodiment 56. An emulsion according to any of embodiments 47-55, wherein at least 50%, e.g., at least 75%, or even at least 90%, of the primary particles of the gelatinized inhibited starch have particle sizes in the range of 0.2-5 microns, e.g., 0.2-2 microns, or 0.2-1 micron.
Embodiment 57. An emulsion according to any of embodiments 47-55, wherein at least 50%, e.g., at least 75%, or even at least 90%, of the primary particles of the gelatinized inhibited starch have particle sizes in the range of 0.5-5 microns, e.g., 0.5-2 microns, or 1-5 microns.
Embodiment 58. An emulsion according to any of embodiments 47-55, wherein the gelatinized inhibited starch is the gelatinization product of a starch having a protein content in the range of 0.2-5 wt % protein, e.g., in the range of 0.2-3 wt % on a dry starch basis.
Embodiment 59. An emulsion according to any of embodiments 47-55, wherein the gelatinized inhibited starch is the gelatinization product of a starch having a protein content in the range of 0.3-8 wt % protein, e.g., in the range of 0.3-5 wt %, or 0.3-3 wt % on a dry starch basis.
Embodiment 60. An emulsion according to any of embodiments 47-55, wherein the gelatinized inhibited starch is the gelatinization product of a starch having a protein content in the range of 0.4-8 wt % protein, e.g., in the range of 0.4-5 wt %, or 0.4-3 wt % on a dry starch basis.
Embodiment 61. An emulsion according to any of embodiments 47-55, wherein the gelatinized inhibited starch is the gelatinization product of a starch having a protein content in the range of 0.5-8 wt % protein, e.g., in the range of 0.5-5 wt %, or 0.5-3 wt % on a dry starch basis.
Embodiment 62. An emulsion according to any of embodiments 47-55, wherein the gelatinized inhibited starch is the gelatinization product of a starch having a protein content in the range of 0.6-8 wt % protein, e.g., in the range of 0.6-5 wt %, or 0.6-3 wt %, or 0.7-8 wt %, or 0.7-5 wt %, or 0.7-3 wt % on a dry starch basis.
Embodiment 63. An emulsion according to any of embodiments 47-55, wherein the gelatinized inhibited starch is the gelatinization product of a starch having a protein content in the range of 1-8 wt % protein, e.g., in the range of 1-5 wt %, or 1-3 wt % on a dry starch basis.
Embodiment 64. An emulsion according to any of embodiments 47-63, wherein the gelatinized inhibited starch is as further described in any of embodiments 27-40.
Embodiment 65. An emulsion according to any of embodiments 46-64, wherein the starch is present in an amount of at least 0.5 wt % of the emulsified phase, e.g., in the range of 0.5-15 wt %, e.g., 0.5-10 wt %, or 0.5-7 wt %, or 1-15 wt %, or 1-10 wt %, or 1-7 wt %.
Embodiment 66. An emulsion according to any of embodiments 46-64, wherein the starch is present in an amount in the range of 0.5-5 wt % of the emulsified phase, e.g., 1-5 wt %.
Embodiment 67. An emulsion according to any of embodiments 46-66, having a median emulsion droplet size in the range of 0.5-100 microns.
Embodiment 68. A foam comprising gas bubbles stabilized within a liquid phase, stabilized by a gelatinized starch according to embodiment 45.
Embodiment 69. A foam comprising gas bubbles stabilized within a liquid phase, stabilized by a gelatinized inhibited starch, wherein the gelatinized inhibited starch has a median primary particle size in the range of 0.2-5 microns and a protein content in the range of 0.2-8 wt. %.
Embodiment 70. A foam according to embodiment 69, wherein the gelatinized inhibited starch is as described in any of embodiments 47-64.
Embodiment 71. A foam according to any of embodiments 68-70, having a median bubble size in the range of 20-3000 microns.
Embodiment 72. A food or beverage product comprising an emulsion or foam according to any of embodiments 46-71.
Embodiment 73. A food or beverage product according to embodiment 72, wherein the food or beverage product is a gravy, a sauce (e.g., a mayonnaise, a white sauce or a cheese sauce), a soup, or a stew.
Embodiment 74. A food or beverage product according to embodiment 72, wherein the food or beverage product is a dressing such as a salad dressing (e.g., pourable or spoonable).
Embodiment 75. A food or beverage product according to embodiment 72, wherein the food or beverage product is a dairy product, e.g. a yogurt, a sour cream, an ice cream or an ice milk.
Embodiment 76. A food or beverage product according to embodiment 72, wherein the food or beverage product is a dairy substitute, e.g., a non-dairy creamer, a plant milk (such as an oat milk, a soy milk or a nut milk) or a food or beverage based thereon (e.g., an ice cream analog based on such milks), or a margarine.
Embodiment 77. A food or beverage product according to embodiment 72, wherein the food or beverage product is a cream filling or a custard.
Embodiment 78. A food or beverage product according to embodiment 72, wherein the food or beverage product is a confectionary, e.g., a chocolate.
Embodiment 79. A food or beverage product according to embodiment 72, wherein the food or beverage product is a mousse.
Embodiment 80. A food or beverage product according to embodiment 72, wherein the food or beverage product is a smoothie or a shake.
Embodiment 81. A personal care product or pharmaceutical product in the form of an emulsion or foam according to any of embodiments 46-64.
Embodiment 82. A method for making an emulsion according to any of embodiments 46-67, or a product according to any of embodiments 72-81 including such an emulsion, comprising mixing the hydrophobic phase, the hydrophilic phase and an inhibited starch according to any of embodiments 1-43 under conditions sufficient to form the emulsion (e.g., under shear).
Embodiment 83. A method making an emulsion according to any of embodiments 47-67, or a product according to any of embodiments 72-81 including such an emulsion, comprising mixing the hydrophobic phase, the hydrophilic phase and an unfragmented inhibited starch having a sedimentation volume in the range of 5-50 mL/g and a protein content in the range of 0.2-8 wt. % under conditions sufficient to fragment the starch to a median primary particle size in the range of 0.2-5 microns and to form the emulsion (e.g., under shear).
Embodiment 84. A method for making a foam according to any of embodiments 68-71, or a product according to any of embodiments 72-81 including such a foam, the method comprising mixing the liquid phase and an inhibited starch according to any of embodiments 1-43 under conditions sufficient to form the foam (e.g., under shear and/or with addition of gas).
Embodiment 85. A method making a foam according to any of embodiments 68-71, or a product according to any of embodiments 72-81 including such a foam, comprising mixing the liquid and an unfragmented inhibited starch having a sedimentation volume in the range of 5-50 mL/g and a protein content in the range of 0.2-8 wt. % under conditions sufficient to fragment the starch to a median primary particle size in the range of 0.2-5 microns and to form the foam (e.g., under shear and/or with addition of gas).
Embodiment 86. A method according to embodiment any of embodiments 82-85, wherein the mixing (e.g., under shear) is performed at a temperature of at least 80° C.
Embodiment 87. A method according to any of embodiments 82-86, wherein the inhibited starch is provided in ungelatinized form, and is gelatinized under the conditions of the mixing (e.g., when performed at temperatures of at least 80° C.).
Embodiment 88. A method according to any of embodiments 82-86, wherein the mixing is performed under conditions that do not gelatinize the starch, but the starch is gelatinized in a later processing step.
Embodiment 89. A method according to any of embodiments 82-86, wherein the inhibited starch is provided in gelatinized form.

1. An inhibited starch having a protein content in the range of 0.2-8 wt % on a dry starch basis.

2. An inhibited starch according to claim 1, having a median primary particle size in the range of 0.2-5 microns.

3. An inhibited starch according to claim 1, wherein the starch is an amaranth starch, a quinoa starch, or a rice starch.

4. An inhibited starch according to claim 1, wherein the starch is a fragmented starch.

5. An inhibited starch according to claim 1, having a median primary particle size in the range of 0.2-2 microns.

6. An inhibited starch according to claim 1, wherein at least 50% of the primary particles of the inhibited starches have particle sizes in the range of 0.2-5 microns.

7. An inhibited starch according to claim 1, having a sedimentation volume in the range of 5-50 mL/g.

8. An inhibited starch according to claim 1, wherein the starch is a fragmented starch, that is inhibited using conditions that provide a sedimentation volume in the range of 5-50 mL/g to a corresponding un-fragmented starch.

9. An inhibited starch according to claim 8, wherein the sedimentation volume is in the range of 5-35 mL/g.

10. An inhibited starch according to claim 1, having a protein content in the range of 0.5-8 wt % protein.

11. (canceled)

12. An emulsion comprising an emulsified phase that is a hydrophobic phase emulsified within a hydrophilic phase, stabilized by a gelatinized product of the inhibited starch according to claim 1.

13. An emulsion comprising an emulsified phase that is a hydrophobic phase emulsified within a hydrophilic phase, stabilized by a gelatinized inhibited starch, wherein the gelatinized inhibited starch has a median primary particle size in the range of 0.2-5 microns and a protein content in the range of 0.2-8 wt. %.

14. An emulsion according to claim 13, wherein the gelatinized inhibited starch is a fragmented starch.

15. An emulsion according to claim 14, wherein the gelatinized inhibited starch is a gelatinized form of a fragmented starch that that is inhibited using conditions that provide a sedimentation volume in the range of 5-50 mL/g to a corresponding un-fragmented starch.

16. An emulsion according to claim 13, wherein the starch is present in an amount in the range of 0.5-5 wt % of the emulsified phase.

17. An emulsion according to claim 13, having a median emulsion droplet size in the range of 0.5-100 microns.

18. A foam comprising gas bubbles stabilized within a liquid phase, stabilized by a gelatinized starch according to claim 1.

19. A foam comprising gas bubbles stabilized within a liquid phase, stabilized by a gelatinized inhibited starch, wherein the gelatinized inhibited starch has a median primary particle size in the range of 0.2-5 microns and a protein content in the range of 0.2-8 wt. %.

20. A food or beverage product comprising an emulsion according to claim 13.

21. A food or beverage product comprising a form according to claim 19.

22. A personal care product or pharmaceutical product in the form of an emulsion according to claim 13.

23. A method making an emulsion according to claim 13 ited starch having a sedimentation volume in the range of 5-50 mL/g and a protein content in the range of 0.2-8 wt. % under conditions sufficient to fragment the starch to a median primary particle size in the range of 0.2-5 microns and to form the emulsion.

24. A method making a foam according to claim 19, comprising mixing the liquid and an unfragmented inhibited starch having a sedimentation volume in the range of 5-50 mL/g and a protein content in the range of 0.2-8 wt. % under conditions sufficient to fragment the starch to a median primary particle size in the range of 0.2-5 microns and to form the foam.

25. A personal care product or pharmaceutical product in the form of a foam according to claim 19.
